SEQUENCE LISTING (6 sequences)

SEQ ID NO: 1 - protein, 15 aa, Artificial sequence
  Synthetic peptide
PVSKMRMATPLLMQA

SEQ ID NO: 2 - protein, 16 aa, Artificial sequence
  Synthetic peptide
CPVSKMRMATPLLMQA

SEQ ID NO: 3 - protein, 15 aa, Artificial sequence
  Synthetic peptide
PVSKMRMACPLLMQA

SEQ ID NO: 4 - protein, 13 aa, Artificial sequence
  Synthetic peptide
PKYVKQNTLKLAT

SEQ ID NO: 5 - protein, 14 aa, Artificial sequence
  Synthetic peptide
CPKYVKQNTLKLAT

SEQ ID NO: 6 - protein, 14 aa, Artificial sequence
  Synthetic peptide
CAGFKGEQGPKGEP

(19) United States
(12) Patent Application Publication
(10) Pub. No.: US 2008/0044405 A1
(43) Pub. Date: Feb. 21, 2008

(54) Noble metal complex-mediated immunosuppression

(75) Inventors: Brian S. DeDecker, Boulder, CO (US); Stephen L. De Wall, Haddonfield, NJ (US)

Correspondence Address: WOLF GREENFIELD & SACKS, P.C., 600 ATLANTIC AVENUE, BOSTON, MA, 02210-2206, US

(73) Assignee: President and Fellows of Harvard College, Cambridge, MA (US)

(21) Appl. No.: 11/710,242

(22) Filed: Feb. 23, 2007

Related U.S. Application Data
(60) Provisional application No. 60/777,204, filed on Feb. 25, 2006.

(51) Int. Cl. A61K 33/24; A61K 39/00; A61K 39/395; A61P 11/06; A61P 37/06; A61P 37/08; G01N 33/50

(52) U.S. Cl. 424/130.1; 424/184.1; 424/646; 424/649; 436/86

(57) ABSTRACT

Certain noble metal complexes are able to strip peptides from class II MHC proteins, resulting in class II MHC peptides in a peptide-empty conformation that do not support T-cell activation. Methods for identifying additional compounds are provided. Also provided are methods for use of this class of compounds to inhibit an immune response and to treat subjects having an unwanted immune response such as autoimmunity, allograft rejection, allergy, asthma, and inflammation.

### RELATED APPLICATION

This application claims benefit under 35 U.S.C. 119(e) of U.S. Provisional Application Ser. No. 60/777,204, filed on Feb. 25, 2006, the entire content of which is incorporated herein by reference.

### BACKGROUND OF THE INVENTION

Class II major histocompatibility complex (MHC) proteins are essential for normal immune system function but also drive many autoimmune responses. They bind peptide antigens in endosomes and present them on the cell surface for recognition by CD4+ T cells (Watts C (1997) Annu Rev Immunol 15:821-50). A small molecule could block an autoimmune response by disrupting MHC-peptide interactions, but this has proven difficult because peptides bind tightly and dissociate slowly from MHC proteins.

### SUMMARY OF THE INVENTION

The invention is based in part on the surprising discovery of a class of noble metal complexes that strip peptides from human class II MHC proteins by an allosteric mechanism (DeDecker BS (2000) Chem Biol 7:R103-7). Biochemical experiments indicate the metal-bound MHC protein adopts a ‘“peptide-empty” conformation that resembles the transition state of peptide loading. Furthermore, these metal inhibitors block the ability of antigen-presenting cells to activate T cells. This previously unknown allosteric mechanism provides a basis for developing a new class of drugs useful for treating and preventing conditions associated with unwanted immune activation.

The discovery of this class of agents is surprising because, although certain gold-containing compounds have been known for years to be useful in the treatment of rheumatoid arthritis, the mechanism of action was not understood and the oxidation state of the gold in such compounds (gold(I)) is different from the oxidation state of gold in the compounds according to the invention (gold(III)).

In one aspect the invention is a method for identifying a candidate immunosuppressive agent. The method according to this aspect of the invention includes the steps of, comprising contacting a peptide—class II MHC complex with a test agent; measuring a test amount of peptide released from the contacted complex; and identifying the test agent as a candidate immunosuppressive agent when the test amount exceeds a control amount of peptide released from the peptide—class II MHC complex in absence of the test agent.

In one embodiment according to this and other aspects of the invention, the peptide is CLIP.

In one embodiment according to this and other aspects of the invention, the test agent comprises a d⁸ noble metal ion in a square-planar oxidated state, wherein two adjacent valencies have exchangeable ligands in cis and two adjacent valencies have non-exchangeable ligands also in cis, forming a four-coordinate complex.

In one embodiment according to this and other aspects of the invention, the test agent comprises Pd(II). In one embodiment according to this and other aspects of the invention, the test agent comprises Pt(II) and excludes cisplatin. In one embodiment according to this and other aspects of the invention, the test agent comprises Au(III).

In one aspect the invention is a method for identifying a candidate immunosuppressive agent. The method according to this aspect of the invention includes the steps of contacting an uncharged class II MHC with a test agent; contacting the uncharged class II MHC with a peptide; measuring a test amount of uncharged class II MHC; and identifying the test agent as a candidate immunosuppressive agent when the test amount exceeds a control amount of uncharged class II MHC in absence of the test agent. In one embodiment the contacting the uncharged class II MHC with the test agent precedes the contacting the uncharged class II MHC with the peptide. In one embodiment the contacting the uncharged class II MHC with the test agent is simultaneous with the contacting the uncharged class II MHC with the peptide.

In one embodiment the measuring comprises measuring an amount of monoclonal antibody bound by the class II MHC, wherein the antibody is specific for a peptide-empty conformation of the class II MHC.

In one embodiment, the test agent comprises a d⁸ noble metal ion in a square-planar oxidated state, wherein two adjacent valencies have exchangeable ligands in cis and two adjacent valencies have non-exchangeable ligands also in cis, forming a four-coordinate complex.

In one aspect the invention is a method for inhibiting an immune response. The method according to this aspect of the invention includes the steps of contacting antigen-presenting cells which express a class II MHC with a composition, other than cisplatin, comprising a d⁸ noble metal ion in a square-planar oxidated state, wherein two adjacent valencies have exchangeable ligands in cis and two adjacent valencies have non-exchangeable ligands also in cis, forming a four-coordinate complex, in an amount effective to inhibit peptide binding by the class II MHC.

In one embodiment according to this and other aspects of the invention the d⁸ noble metal ion is Pd(II). In one embodiment according to this and other aspects of the invention the d⁸ noble metal ion is Pt(II). In one embodiment according to this and other aspects of the invention the d⁸ noble metal ion is Au(III).

The invention in one aspect is a method for treating a subject having or at risk of developing an unwanted immune response. The method according to this aspect of the invention includes the step of administering to the subject a composition, other than cisplatin, comprising a d⁸ noble metal ion in a square-planar oxidated state, wherein two adjacent valencies have exchangeable ligands in cis and two adjacent valencies have non-exchangeable ligands also in cis, forming a four-coordinate complex, in an amount effective to inhibit the unwanted immune response.

In one embodiment according to this aspect of the invention the unwanted immune response is an autoimmune disease. In one embodiment according to this aspect of the invention the autoimmune disease is selected from the group consisting rheumatoid arthritis, systemic lupus erythematosus, insulin-dependent diabetes mellitus, multiple sclerosis, pemphigus vulgaris, gluten-sensitive enteropathy, and chronic active hepatitis.

In one embodiment according to this aspect of the invention the unwanted immune response is allograft rejection.

In one embodiment according to this aspect of the invention the unwanted immune response is an allergy.

In one embodiment according to this aspect of the invention the unwanted immune response is asthma.

In one embodiment according to this aspect of the invention the unwanted immune response is inflammation.

In one embodiment according to this aspect of the invention the administering is systemically administering. In one embodiment according to this aspect of the invention the administering is locally administering to a site involved in the unwanted immune response.

In one embodiment according to this aspect of the invention the method further includes the step of administering to the subject an immunosuppressive agent selected from the group consisting of steroids, non-steroidal anti-inflammatory drugs, immunosuppressive antibodies, cyclosporines, methotrexate, azathioprine, mycophenolate mofetil, tacrolimus, and sirolimus.

In one embodiment according to this aspect of the invention the method further includes the step of contacting the subject with an antigen. In one embodiment the contacting the subject is administering to the subject.

According to another aspect of the invention, a composition of matter is provided. The composition is isolated soluble fragment of class II MHC bound to a d⁸ noble metal ion in a square-planar oxidated state, wherein two adjacent valencies have exchangeable ligands in cis and two adjacent valencies have non-exchangeable ligands also in cis, forming a four-coordinate complex. The composition can be used for a variety of purposes as disclosed herein as an inhibitor of DM, and also for the purpose of raising antibodies to empty class II MHC. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1a is a bar graph depicting the ability of indicated metal complexes to disrupt the MHC-peptide interaction. HA peptide, hemagglutinin peptide.

FIG. 1b is graph depicting the ability of the indicated metal complexes to cause release of CLIP peptide from DR1-CLIP complex in a concentration-dependent manner.

FIG. 1c is a graph depicting peptide sequence-, affinity-, and pH-independence of the ability of cisplatin to displace peptide from DR1.

FIG. 2a is a graph depicting the rapid dissociation of peptide according to the indicated metal complex.

FIG. 2b is a graph depicting the effect of DM on dissociation kinetics of CLIP.

FIG. 2c is a bar graph depicting the pH-independence of peptide dissociation in the presence of the indicated metal complexes.

FIG. 2d is a bar graph depicting peptide bound in the presence of cisplatin or CLIP, in the presence or absence of DM.

FIG. 3a is a pair of graphs depicting the results of Biacore binding of indicated proteins and complexes with an antibody specific for peptide-empty DR1.

FIG. 3b is a graph depicting circular dichroism spectra of the indicated proteins and complexes.

FIG. 3c is a graph depicting gel filtration chromatography of the indicated complexes (upper panel) and corresponding western blots for various fractions (lower panel).

FIG. 4a is a graph depicting peptide dissociation from the indicated complexes as a function of concentration of KAuCl₄.

FIG. 4b is a bar graph depicting in vitro activation of sodium Au(I)-thiomalate by hypochlorite (OC1⁻).

FIG. 4c is a bar graph depicting inhibition of T-cell activation by APCs treated with indicated metal complexes. Untreated APCs mixed with treated APCs, similar to untreated APCs alone, strongly induce T-cell activation.

### DETAILED DESCRIPTION OF THE INVENTION

The present invention relates at least in part to using certain noble metal ion-containing compounds to inhibit an immune response. It has been discovered according to the invention that certain d⁸ noble metal ions capable of forming square-planar, four-coordinate complexes can form a complex with class II MHC molecules and interfere with the formation of complexes between class II MHC molecules and peptides that normally can bind to the peptide-binding cleft of the class II MHC molecules. It has also been discovered according to the invention that certain d⁸ noble metal ions capable of forming square-planar, four-coordinate complexes can form a complex with peptide-class II MHC complexes and cause bound peptide to dissociate from peptide—class II MHC complexes. The resulting class II MHC molecules are unable to present antigen and thus do not induce T-cell activation. Accordingly, in one aspect the invention provides methods of treating a subject having or at risk of developing an unwanted immune response. An unwanted immune response includes, without limitation, an autoimmune disease, inflammation, allograft rejection, allergy, and asthma.

The present invention also relates at least in part to methods for screening for certain noble metal ion-containing compounds that may be useful to inhibit an immune response. In one embodiment the screening methods are based on the discovery that certain d⁸ noble metal ions capable of forming square-planar, four-coordinate complexes can form a complex with class II MHC molecules and interfere with the formation of complexes between class II MHC molecules and peptides that normally can bind to the peptide-binding cleft of the class II MHC molecules. In one embodiment the screening methods are based on the discovery that certain d⁸ noble metal ions capable of forming square-planar, four-coordinate complexes can form a complex with peptide—class II MHC complexes and cause bound peptide to dissociate from peptide—class II MHC complexes.

As disclosed in greater detail below, d⁸ noble metal ions capable of forming square-planar, four-coordinate complexes useful according to the invention include but are not limited to palladium(II), platinum(II), and gold(III), also referred to herein as Pd^{II}, Pt^{II}, and Au^{III}, respectively. In one embodiment the noble metal ions are soft Lewis acids that favor coordination by soft Lewis bases such as the sulfur atoms found in cysteine, methionine, and cystine residues. In one embodiment the noble metal ions form square-planar, four-coordinate complexes in which at least two available (e.g., exchangeable) ligands are positioned in cis with respect to each other. As an example of such embodiment, Pt^{II} forms a square-planar, four-coordinate complex with two exchangeable chloride ions in cis and two amine groups in cis in cis-diamminedichloroplatinum (II), commonly known as cisplatin. It has been discovered according to the present invention that while cisplatin is active as an inhibitor of peptide-class II MHC complex formation, transplatin is not. In transplatin Pt^{II} forms a square-planar, four-coordinate complex with two exchangeable chloride ions in trans and two amine groups in trans.

In one embodiment the noble metal ions form square-planar, four-coordinate complexes in which there are two available (e.g., exchangeable) ligands positioned in cis with respect to each other, such as there are in cisplatin.

The exchangeable ligands can be identical or they can be non-identical.

When there are two non-exchangeable ligands, in one embodiment the non-exchangeable ligands are the same. In another embodiment in which there are two non-exchangeable ligands, the non-exchangeable ligands are different.

It has also been discovered according to the present invention that a non-exchangeable ligand, if present, can be substituted with another non-exchangeable ligand. Selection and/or synthesis of test compounds for use in the screening methods of the invention can be based on the principle of substituting one or more non-exchangeable ligands from one test compound to another. Alternatively or in addition, selection and/or synthesis of test compounds for use in the screening methods of the invention can be based on the principle of substituting one or more exchangeable ligands from one test compound to another.

It has also been discovered according to the present invention that a non-exchangeable ligand, if present, can be replaced by an exchangeable ligand. An added group could add more contacts with the MHC that would increase its affinity for MHC and/or strengthen the complex with DM. Selection and/or synthesis of test compounds for use in the screening methods of the invention can be based on the principle of substituting one or more exchangeable ligands for non-exchangeable ligands from one test compound to another.

Exchangeable ligands can include, without limitation, the following: halide (F, Cl, Br, I) sulfate, phosphate, nitrate, or anionic-charged organic species. The anionic charged species can be sulfonate or carboxylate. The sulfonate can be mesylate, besylate, tosylate, and the like. The carboxylate can be formate, acetate, citrate, fumerate and the like.

Non-exchangeable ligands can include, without limitation, any organic molecule attached to the metal atom, preferably via a nitrogen or sulfur. It also is possible that one, but not both, of the non-exchangeable ligands can be substituted with a hydrogen or an exchangeable ligand. Examples of organic molecules are those constructed of C, N, S, P, O, and H. Organic molecules include amino acids, amino acid analogs, nucleic acids, nucleic acid analogs, and polymers thereof, and carbon containing molecules, typically C₁-C₃₀, substituted or not (typically with H, halo, OH, lower alkyl, lower alkoxy, or phenyl), including, but not limited to, alkyl, alkenyl, alkynyl, aryl, aryl substituted alkyl, or heteroaryl.

More specific examples attached via an N or an S include N(CH₂)₀₋₄ or S(CH2)₀₋₄ attached to R, —OR, —SR, —NR₂, —ONR₂, —NO₂, —CN, —C(O)R, —C(S)R, —C(O)OR, —C(S)OR, —C(O)SR, —C(S)SR, —C(O)NR₂, —C(S)NR₂, —C(O)NR(OR), —C(S)NR(OR), —C(O)NR(SR), C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, where each R is independently selected from the group consisting of —H, (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl and (C₁-C₆) alkoxy, the aryl substituents are each independently selected from the group consisting of -halo, trihalomethyl, —R, —R′, —OR′, —SR′, NR′₂, —NO₂, —CN, —C(O)R′, —C(S)R′, —C(O)OR′, —C(S)OR′, —C(O)SR′ and —C(S)SR′;

the alkyl, alkenyl and alkynyl substituents are each independently selected from the group consisting of -halo, —R′, —OR′, —SR′, NR′₂, —NO₂, —CN, —C(O)R′, —C(S)R′, —C(O)OR′, —C(S)OR′, —C(O)SR′, —C(S)SR′, aryl, γ-butyrolactonyl, pyrrolidinyl and succinic anhydridyl; and

each R′ is independently selected from the group consisting of —H, (C₁-C₆) alkyl, (C₁-C₆) alkenyl and (C₁-C₆) alkynyl.

“Alkyl”, in general, refers to an aliphatic hydrocarbon group which may be straight, branched or cyclic having from 1 to about 10 carbon atoms in the chain, and all combinations and subcombinations of ranges therein. “Branched” refers to an alkyl group in which a lower alkyl group, such as methyl, ethyl or propyl, is attached to a linear alkyl chain. In certain preferred embodiments, the alkyl group is a C₁-C₅ alkyl group, i.e., a branched or linear alkyl group having from 1 to about 5 carbons. In other preferred embodiments, the alkyl group is a C₁-C₃ alkyl group, i.e., a branched or linear alkyl group having from 1 to about 3 carbons. Exemplary alkyl groups include methyl, ethyl, n-propyl, isopropyl, butyl, isobutyl, sec-butyl, tert-butyl, pentyl, hexyl, heptyl, octyl, nonyl and decyl. “Lower alkyl” refers to an alkyl group having 1 to about 6 carbon atoms. Preferred alkyl groups include the lower alkyl groups of 1 to about 3 carbons.

“Alkenyl”, in general, refers to an alkyl group containing at least one carbon—carbon double bond and having from 2 to about 10 carbon atoms in the chain, and all combinations and subcombinations of ranges therein. In certain preferred embodiments, the alkenyl group is a C₂-C₁₀ alkyl group, i.e., a branched or linear alkenyl group having from 2 to about 10 carbons. In other preferred embodiments, the alkenyl group is a C₂-C₆ alkenyl group, i.e., a branched or linear alkenyl group having from 2 to about 6 carbons. In still other preferred embodiments, the alkenyl group is a C₃-C₁₀ alkenyl group, i.e., a branched or linear alkenyl group having from about 3 to about 10 carbons. In yet other preferred embodiments, the alkenyl group is a C₂-C₅ alkenyl group, i.e., a branched or linear alkenyl group having from 2 to about 5 carbons. Exemplary alkenyl groups include, for example, vinyl, propenyl, butenyl, pentenyl, hexenyl, heptenyl, octenyl, nonenyl and decenyl groups.

“Alkoxy”, in general, refers to an alkyl-O— group where alkyl is as previously described. Exemplary alkoxy groups include, for example, methoxy, ethoxy, propoxy, butoxy and heptoxy.

“Aryl”, in general, refers to an aromatic carbocyclic radical containing 6, 10 or 14 carbons. The phenyl group may be optionally substituted with one or two or more substituents. Preferred aryl group substituents include alkyl groups, preferably C₁-C₅ alkyl groups. Exemplary aryl groups include phenyl and naphthyl.

“Aryl-substituted alkyl”, in general, refers to an linear alkyl group, preferably a lower alkyl group, substituted at a carbon with an optionally substituted aryl group, preferably an optionally substituted phenyl ring. Exemplary aryl-substituted alkyl groups include, for example, phenylmethyl, phenylethyl and 3-(4-methylphenyl)propyl.

It should be noted that the metal need not cross a membrane in order to work. As described above, normally the antigen (and presumably the metal compound) traffic into endosome, where peptide loading normally takes place. The loaded MHC is then transported back to the cell surface. The metal complex can work at any of these steps and locations and thus may but need not cross the membrane in order to exert its effect.

Compounds useful according to the invention that do not cross the cell membrane can be prepared by adding charged groups and by increasing size. For example, 5-amino-1,10-phenanthroline can be used as a ligand with any of the active metal ions. It is possible to attach groups to the phenanthroline by coupling to the 5-amino group. Moieties that add charge can include, for example, betaine (a quaternary amine that adds positive charge) which can be coupled to the 5-amino group of phenanthroline through an amide bond, and taurine (a sulfonic acid that adds a negative charge) which can be coupled to the 5-amino group of phenanthroline via glutaric acid (a dicarboxylic acid, a homo bifunctional crosslinker). Groups that add bulk and improve stability can include polyethylene glycol (PEG), e.g., PEG-NHS esters coupled via an amide bond to the 5-amino group of the phenanthroline.

Phenanthroline is just one example of a class of molecules that can be used to increase charge and/or size. It is possible to use any other ligand that has a free amine “handle” for coupling these types of groups. In addition, it is possible to couple almost any carboxylic acid or primary alkyl or benzyl halide to this type of amine.

Alternatively or in addition, ligands can be chosen to provide more protein contacts to increase specificity and affinity for class II MHC proteins. For example, using routine methods it is possible to synthesize a combinatorial library of compounds (e.g., peptides) that are coupled to a variety of ligands that can coordinate the active metal ions. See, for example, Robillard M S et al. (2003) J Comb Chem 5:821-5.

As yet a further alternative, it may be possible to use “cage” molecules activated by oxidation. One type of ligand that may work are a class of ligands called cryptands. A cryptand is a molecular entity comprising a cyclic or polycyclic assembly of binding sites that contains three or more binding sites held together by covalent bonds, and which defines a molecular cavity in such a way as to bind (and thus “hide” in the cavity) another molecular entity, the guest (a cation, an anion or a neutral species), more strongly than do the separate parts of the assembly (at the same total concentration of binding sites). The adduct thus formed is called a “cryptate”. The term is usually restricted to bicyclic or oligocyclic molecular entities. There are probably hundreds to thousands of different cryptands of varying sizes and containing different functional groups such as disulfides. A cryptand with a disulfide in it might be used as the part that is cleaved to generate the active drug.

A class II MHC molecule as used herein has its usual meaning as well recognized in the art. Major histocompatibility complexes are heterodimeric proteins that are involved in presenting processed antigen to cells of the immune system. There are two classes of such complexes, referred to as class I and class II. Class I MHC are widely expressed on the surface of most nucleated cells in mammals, and they generally present peptide antigens derived from proteins of cytosolic (endogenous) origin, i.e., generally self-antigens of the host. When they are loaded with antigen (charged), class I MHC can interact with T-cell antigen receptor (TCR) expressed on the surface of CD8+ T lymphocytes (T cells). Under certain conditions, CD8+ T cells become activated in connection with presentation of antigen in the context of class I MHC, leading to cytolytic activity directed against target cells.

In contrast, class II MHC are normally expressed on specialized cells of the immune system called antigen-presenting cells (APCs) and endothelium, and they generally present peptide antigens derived from proteins of extracellular (exogenous) origin, i.e., generally non-self-antigens.

The generation of class II MHC-associated peptides from endocytosed antigens involves proteolytic degradation of internalized proteins in endocytic vesicles, followed by binding of processed antigen (i.e., peptides) to class II MHC molecules within the vesicles. Class II MHC molecules are synthesized in the endoplasmic reticulum and are transported to endosomes with an associated protein (invariant chain, Ii) occupying the peptide-binding cleft of newly synthesized class II MHC molecules. Exocytic vesicles transporting the class II MHC molecules from the endoplasmic reticulum fuse with endocytic vesicles containing processed antigen. Within the fused vesicles, proteolytic enzymes degrade the invariant chain to leave a 24-amino acid remnant peptide called class II-associated invariant chain peptide (CLIP) occupying the peptide-binding cleft of the class II MHC molecule. In order to load class II MHC with peptide from processed antigen, a catalytic molecule called HLA-DM (herein, simply DM) facilitates exchange of peptide for CLIP. Class II MHC molecules are believed to be stabilized by bound peptide, and the resulting peptide—class II MHC complexes are delivered to the surface of the APC, where they are displayed for recognition by CD4+ T cells.

When they are loaded with antigen (charged), class II MHC can interact with TCR expressed on the surface of CD4+ T cells. Under certain conditions CD4+ T cells become activated in connection with presentation of antigen in the context of class II MHC, leading to secretion of cytokines and activation of phagocytes and B lymphocytes.

Methods of the invention can be used to identify a candidate immunosuppressive agent. As used herein, a candidate immunosuppressive agent is any agent that reduces binding between a peptide and a class II MHC molecule and may be used to reduce or prevent an immune response. In one embodiment the candidate immunosuppressive agent binds to a class II MHC molecule at a site other than the peptide-binding cleft of the class II MHC molecule. In one embodiment the candidate immunosuppressive agent is a composition that includes a d⁸ noble metal ion capable of forming a square-planar, four-coordinate complex, as disclosed herein.

In one aspect of the invention a preformed peptide—class II MHC complex is contacted with a test agent and the amount of peptide released from the complex is then measured. A peptide—class II MHC complex as used herein refers to a molecular complex formed through association of a peptide with a class II MHC molecule, wherein the peptide occupies the peptide-binding cleft of the class II MHC molecule.

Conditions for the contacting step are selected to be appropriate for permitting the various assay components to interact. Such conditions in one embodiment are physiologic conditions, e.g., 37° C. in a suitable buffer or cell culture medium. In one embodiment the conditions are in vitro. In one embodiment the conditions are in vivo. The conditions can also include permitting the assay components to interact for a predetermined or definite amount of time. Those skilled in the art will be able to identify and optimize conditions suitable for the contacting step.

The amount of peptide released is compared to a corresponding amount of peptide released when no test agent is included in the assay. Alternatively, the amount of peptide released is compared to a corresponding amount of peptide released when a reference agent is included in the assay. In either embodiment, a test result obtained with a test agent is compared with a control result obtained without the test agent or with the reference agent. The control result can be a historical control result or it can be a control result obtained in parallel with obtaining the test result.

The assay can include one or more steps to isolate excess reactants from products. The assay can also include one or more additional components or steps, e.g., to assist with the detection of released peptide, peptide—class II MHC complex, and/or class II MHC.

The assay includes the step of measuring an amount of peptide released from the peptide—class II MHC complex. Any suitable method of measurement can be used. In one embodiment the peptide is labeled with a tag that can be measured either directly or indirectly. For example, the peptide can be labeled with a fluorescent tag that can be detected with a suitable photodetector. In one embodiment the peptide can be labeled with a radionuclide tag that can be detected with a suitable detector. In one embodiment the peptide is tagged with a suitable fluorophore and is measured using fluorescence polarization, as described in greater detail in the Examples below. In one embodiment the peptide is untagged and is measured, e.g., using a suitable peptide-specific enzyme-linked immunosorbent assay (ELISA).

As an alternative to measuring released peptide, in another embodiment the remaining bound peptide can be measured. It is believed, however, that measurement of released peptide will generally be more sensitive than measurement of remaining bound peptide.

In one aspect of the invention an uncharged class II MHC molecule is contacted with a test agent, either prior to or at least substantially coincident with contacting the uncharged class II MHC molecule with a peptide that is capable of forming a complex with the class II MHC molecule (as described above), and the amount of remaining uncharged class II MHC molecule is then measured. As used herein, an uncharged class II MHC molecule refers to a class II MHC molecule with an empty peptide-binding cleft. The term “charged” as used in this context does not describe or relate to an electric charge on or of the class II MHC molecule.

Conditions for the contacting step or steps are selected according to principles as outlined above. The assay can include one or more steps to isolate excess reactants from products. The assay can also include one or more additional components or steps, e.g., to assist with the detection of remaining uncharged class II MHC molecule.

The amount of remaining uncharged class II MHC is compared to a corresponding amount of remaining uncharged class II MHC when no test agent is included in the assay. Alternatively, the amount of remaining uncharged class II MHC is compared to a corresponding amount of remaining uncharged class II MHC when a reference agent is included in the assay. In either embodiment, a test result obtained with a test agent is compared with a control result obtained without the test agent or with the reference agent. The control result can be a historical control result or it can be a control result obtained in parallel with obtaining the test result.

It has been discovered according to the invention that active agents of the invention induce class II MHC molecules to adopt a peptide-empty conformation. A peptide-empty conformation as used herein refers to a three-dimensional conformation of a class II MHC molecule in which the peptide-binding cleft of the class II MHC molecule is unoccupied. It is generally believed that binding of peptide within the peptide cleft is accompanied by a conformational change in the class II MHC molecule. This peptide-empty conformation can be detected, for example, with an antibody that is specific for such conformation. As described in greater detail below, one such antibody is MEM-264, described in Carven G J et al. (2004) J Biol Chem 279:16561-70.

The assay includes the step of measuring an amount of remaining uncharged class II MHC. Any suitable method of measurement can be used. In one embodiment the remaining uncharged class II MHC is contacted with an antibody that specifically binds peptide-empty conformation and binding can be measured either directly or indirectly. For example, the class II MHC molecule or test agent bound to the class II MHC molecule can be labeled with a fluorescent tag that can be detected with a suitable photodetector, and the class II MHC molecule is captured and detected on a surface coated with antibody that specifically binds peptide-empty conformation. In one embodiment the class II MHC molecule or test agent bound to the class II MHC molecule can be labeled with a radionuclide tag that can be detected with a suitable detector.

In one embodiment the remaining uncharged class II MHC molecule is isolated from the test agent, including test agent bound to the class II MHC molecule, and then the amount of a peptide subsequently bound by the resulting class II MHC molecule is measured. In this embodiment the peptide bound by the resulting class II MHC molecule can be labeled so that the amount of subsequently bound peptide is correlated with the amount of previously remaining uncharged class II MHC.

In one embodiment the peptide is specific for the particular class II MHC molecule. For example, in one embodiment the peptide is a hemagglutinin (HA) peptide and the class II MHC molecule is capable of binding the HA peptide. As another example, in one embodiment the peptide is an ovalbumin (OVA) peptide and the class II MHC molecule is capable of binding the OVA peptide. Those of ordinary skill in the art will be familiar with other peptides and class I MHC molecules suitable for use together.

In another embodiment the peptide is not specific for the particular class II MHC molecule. For example, in one embodiment the peptide is a CLIP peptide. As another example, in one embodiment the peptide is DM or a class II MHC-binding fragment thereof.

In these and other aspects of the invention, a peptide, class II MHC molecule, peptide—class II MHC complex, and/or cell is an isolated peptide, class II MHC molecule, peptide—class II MHC complex, and/or cell. The term isolated as used herein means removed from other components, such as may be found in nature. For example, an isolated class II MHC molecule in one embodiment is a class II MHC molecule apart from a cell. In another embodiment an isolated class II MHC molecule is a class II MHC molecule expressed by an isolated cell. The term isolated as used herein can but need not mean purified.

Test agents according to the invention can include known compounds, novel compounds, or a combination of known and novel compounds. In one embodiment the test agent is a composition that includes a d⁸ noble metal ion capable of forming a square-planar, four-coordinate complex. As disclosed herein, examples of test agents include cisplatin (Pt^{II}(NH₃)₂Cl₂), carboplatin (diammine[1,1-cyclobutane-dicarboxylato(2-)-O,O′]—Pt^{II}), K₂Pt^{II}Cl₄, K₂Pd^{II}Cl₄, and KAu^{III}Cl₄. These same exemplary test agents can also be used as reference compounds in methods of the invention wherein an effect of a test compound is compared against a corresponding effect of a reference compound, e.g., to assess relative potency.

In one embodiment the test agent is a composition that includes a d⁸ noble metal ion capable of forming a square-planar, four-coordinate complex, having at least two exchangeable ligands in cis to one another.

In one embodiment the test agent is a composition that includes a d⁸ noble metal ion capable of forming a square-planar, four-coordinate complex, having two exchangeable ligands in cis to one another.

In one embodiment the test agent is a composition that includes a d⁸ noble metal ion capable of forming a square-planar, four-coordinate complex, having at least two exchangeable ligands in cis to one another and at least one non-exchangeable ligand.

In one embodiment the test agent is a composition that includes a d⁸ noble metal ion capable of forming a square-planar, four-coordinate complex, having at two exchangeable ligands in cis to one another and two non-exchangeable ligands in cis to one another.

In one embodiment a test agent is a composition that includes a metal ion, other than a d⁸ noble metal ion, capable of forming a square-planar, four-coordinate complex.

In one embodiment the test agent is a composition that includes a metal ion, other than a d⁸ noble metal ion, capable of forming a square-planar, four-coordinate complex, having at least two exchangeable ligands in cis to one another.

In one embodiment the test agent is a composition that includes a metal ion, other than a d⁸ noble metal ion, capable of forming a square-planar, four-coordinate complex, having two exchangeable ligands in cis to one another.

In one embodiment the test agent is a composition that includes a metal ion, other than a d⁸ noble metal ion, capable of forming a square-planar, four-coordinate complex, having at least two exchangeable ligands in cis to one another and at least one non-exchangeable ligand.

In one embodiment the test agent is a composition that includes a metal ion, other than a d⁸ noble metal ion, capable of forming a square-planar, four-coordinate complex, having at two exchangeable ligands in cis to one another and two non-exchangeable ligands in cis to one another.

In one embodiment a composition that includes a d⁸ noble metal ion according to the invention is Au^{III}.(4,4′-bipyridyl)(OH)₂.

In one aspect the invention provides a method for inhibiting an immune response. Antigen-presenting cells (APCs) expressing a class II MHC protein are contacted with a noble metal ion-containing composition of the invention, resulting in stripping any bound peptide from the class II MHC protein and preventing loading of any uncharged class II MHC protein with peptide, thereby rendering the APCs incapable of activating CD4+ T cells. In one embodiment the method is performed in vitro. In one embodiment the method is performed in vivo.

In one embodiment the noble metal ion-containing composition is not cisplatin.

In one embodiment the noble metal ion-containing composition is not a gold(I)-containing composition.

In one embodiment the noble metal ion-containing composition is not a gold(I) or a gold(III)-containing composition.

The method may be of particular use in the setting of contact between an immune cell (or a subject) and a particular antigen, e.g., an allergen or a transplant antigen. The method used in this setting can be used to treat or to prevent the development of an immune response to the antigen. The antigen can be one that is already in contact with the immune cell, for example in an existing autoimmune disease or an existing allergic reaction. Alternatively and in addition, the antigen can be one that is expected or planned to come in contact with the immune cell or subject in the future. For example, the composition can be administered to a subject with a seasonal allergic condition such as hay fever, prior to seasonal encounter with allergen.

In one aspect the invention provides a method for treating a subject having or at risk of developing an unwanted immune response. A subject as used herein refers to a mammal. In one embodiment the subject is a human. As used herein, treat and treating refer to any measure that results in preventing the onset of, slowing the progression of, ameliorating, or eliminating a condition or at least one symptom associated with a condition in a subject.

An unwanted immune response as used herein refers to any immune response in a subject that is associated with an undesirable immune-mediated condition. Undesirable immune-mediated conditions include but are not limited to autoimmune disease, allograft rejection, allergy, asthma, and inflammation.

As used herein a subject having an unwanted immune response is a subject with an existing unwanted immune response. As used herein a subject at risk of developing an unwanted immune response is a subject without symptoms of an existing unwanted immune response but is prone to developing such immune response. A subject can be prone to developing an unwanted immune response for any of a variety of reasons, including but not limited to genetic predisposition (e.g., having a particular HLA-DR antigen associated with autoimmune disease), anticipated encounter with an allergen to which the subject has an allergy, planned transplantation.

In one embodiment the method further includes the step of contacting the subject with an antigen. The contacting according to this embodiment can be either passive, e.g., through environmental exposure, or active, e.g., through administration.

Cytokines are secreted proteins and glycoproteins that are produced by many different cell types, including cells of the immune system, and mediate inflammatory and antigen-specific immune reactions. Cytokines are principal soluble mediators of communication between cells of the immune system. Cytokines include a number of classes of molecules, including but not limited to interleukins, interferons, chemokines, tumor necrosis factors, transforming growth factors, and colony stimulating factors. Interleukins include at least 30 proteins specifically including but are not limited to interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 14 (IL-14), interleukin 15 (IL-15), interleukin 16 (IL-16), interleukin 17 (IL-17), interleukin 18 (IL-18), etc. Interferons specifically include but are not limited to interferon alpha (IFN-α), of which there are several isoforms, interferon beta (IFN-β), of which there are at least two isoforms, and interferon gamma (IFN-γ). Tumor necrosis factors specifically include but are not limited to tumor necrosis factor alpha (TNF-α). Chemokines specifically include but are not limited to IP-10, RANTES, I-TAC, MIP-1α, and MIP-1. Transforming growth factors specifically include but are not limited to transforming growth factor beta (TGF-β).

Certain cytokines are associated with T helper type 1 (Th1) immune responses, and certain other cytokines are associated with T helper type 2 (Th2) immune responses. These two types of immune responses are generally believed to be associated with different clinical manifestations, and they are believed to be cross-regulatory. For example, Th1-associated immune responses are characterized by cell-mediated immunity, such as occurs in tumor killing, certain aspects of allograft rejection, and certain forms of autoimmune disease. Th1-associated immune responses are also characterized by a predominance of IFN-γ, IL-2, and IL-12. Th2-associated immune responses include humoral immunity, such as occurs in allergy, allergic asthma, certain aspects of allograft rejection, and certain forms of autoimmune disease. Th2-associated immune responses are also characterized by a predominance of IL-4, IL-5, IL-10, and immunoglobulin E (IgE).

In humans class II MHC are polymorphic and include so-called HLA-DR, -DP, and -DQ antigens. Certain of these antigens are associated with increased risk for having or developing certain autoimmune and other diseases. For example, rheumatoid arthritis is associated with Dw4/DR4; systemic lupus erythematosus (lupus; SLE) is associated with DR3, DR2, and DQ3; insulin-dependent diabetes mellitus (type 1 diabetes mellitus) is associated with DR3 and DR4; multiple sclerosis is associated with DR2; pemphigus vulgaris is associated with DR4; gluten-sensitive enteropathy is associated with DR3; and chronic active hepatitis is associated with DR3.

The active agents according to the invention are useful for treating and preventing autoimmune disease. Autoimmune disease is a class of diseases in which an subject's own antibodies react with host tissue or in which immune effector T cells are autoreactive to endogenous self peptides and cause destruction of tissue. Thus an immune response is mounted against a subject's own antigens, referred to as self-antigens. Autoimmune diseases include but are not limited to rheumatoid arthritis, insulin-dependent diabetes mellitus, inflammatory bowel disease, multiple sclerosis, systemic lupus erythematosus, autoimmune encephalomyelitis, myasthenia gravis, Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, and Sjögren's syndrome.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer.

The compositions and methods of the invention are useful in connection with the treatment of various autoimmune diseases, including but not limited to rheumatoid arthritis; SLE; insulin-dependent diabetes mellitus; multiple sclerosis; pemphigus vulgaris; gluten-sensitive enteropathy; and chronic active hepatitis. These and other autoimmune diseases are well known in the medical arts.

Class II-restricted immune responses can also include or contribute to conditions characterized by unwanted immune response such as allograft rejection, graft-versus-host disease (GvHD), allergy, asthma, and inflammation. The compositions and methods of the invention thus are useful in the treatment of these additional conditions characterized by unwanted immune response, including allograft rejection, graft-versus-host disease (GvHD), allergy, asthma, and inflammation.

Allograft rejection refers to acute or chronic immune-mediated injury to a cell, tissue, or organ that has been transplanted from a donor individual to a recipient individual. The rejection results in disruption of functional, anatomical, or both functional and anatomical features of the graft cell, tissue, or organ. Such changes can range from subtle change to death of the graft cell, tissue, or organ.

An antigen as used herein refers to a molecule capable of provoking an immune response. Antigens include but are not limited to cells, cell extracts, proteins, polypeptides, peptides, nucleic acids, polysaccharides, polysaccharide conjugates, peptide and non-peptide mimics of polysaccharides and other molecules, small molecules, lipids, glycolipids, carbohydrates, viruses and viral extracts and multicellular organisms such as parasites and allergens. The term antigen broadly includes any type of molecule which is recognized by a host immune system as being foreign.

Antigens include allergens, cancer antigens, microbial antigens, and, in the context of autoimmune disease, self-antigens.

A cancer antigen as used herein is a compound, such as a peptide or protein, associated with a tumor or cancer cell surface and which is capable of provoking an immune response when expressed on the surface of an antigen presenting cell in the context of an MHC molecule. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A microbial antigen as used herein is an antigen of a microorganism and includes but is not limited to virus, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

An allergy refers to an acquired hypersensitivity to an allergen. A subject having an allergy is a subject that has an allergic reaction in response to exposure to or contact with an allergen. A subject having an allergy includes a subject without symptoms of allergy in absence of exposure to or contact with an allergen.

An allergen refers to a substance (e.g., an antigen) that can induce an allergic or asthmatic response in a susceptible subject. The list of allergens is enormous and can include pollens, insect venoms, animal dander dust, fungal spores and drugs (e.g., penicillin). Examples of natural, animal and plant allergens include but are not limited to proteins specific to the following geniuses: Canis (Canis familiaris); Dermatophagoides (e.g., Dermatophagoides farinae); Felis (Felis domesticus); Ambrosia (Ambrosia artemiisfolia; Lolium (e.g., Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g., Plantago lanceolata); Parietaria (e.g., Parietaria officinalis or Parietaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis and Juniperus ashei); Thuya (e.g., Thuya orientalis); Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis (e.g., Dactylis glomerata); Festuca (e.g., Festuca elatior); Poa (e.g., Poa pratensis or Poa compressa); Avena (e.g., Avena sativa); Holcus (e.g., Holcus lanatus); Anthoxanthum (e.g., Anthoxanthum odoratum); Arrhenatherum (e.g., Arrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis); and Bromus (e.g., Bromus inermis).

As used herein, “asthma” refers to a disorder of the respiratory system that is episodic and characterized by inflammation with narrowing of the airways and increased reactivity of the airways to inhaled agents. Asthma is frequently, although not exclusively associated with atopic or allergic symptoms. Symptoms of asthma are widely recognized to include dyspnea, cough, and wheezing; while all three symptoms typically coexist, their coexistence is not required to make a diagnosis of asthma.

A “subject having asthma” as used herein refers to a subject with an existing acute exacerbation of asthma, either new-onset or recurrent, or a history of asthma, or a known or suspected predisposition toward developing asthma. A subject having asthma thus can have active asthma or can be asymptomatic and between acute exacerbations. In one embodiment a subject having asthma is a subject having asthma that is associated with allergic symptoms, i.e., allergic asthma.

Active agents of the invention can be used in combination with other therapeutic agents, including antigens. The active agent and one or more other therapeutic agents may be administered simultaneously or sequentially. When the one or more other therapeutic agents are administered simultaneously, they can be administered in the same or separate formulations, but are administered at the same time. When the one or more other therapeutic agents are administered sequentially, they can be administered in the same or separate formulations, but are administered at different times. The separation in time between the administration of these compounds may be a matter of minutes or it may be longer. In one embodiment the active agent is administered before administration of the one or more other therapeutic agents. In one embodiment the active agent is administered following administration of the one or more other therapeutic agents.

Compositions of the invention can be used alone or in combination with each other and/or with other immunosuppressive agents. Immunosuppressive agents are well known in the art and include, without limitation, steroids, non-steroidal anti-inflammatory drugs (NSAIDs), immunosuppressive antibodies, cyclosporines, methotrexate, Additional immunosuppressive agents can include certain cytokines, antibodies specific for certain cytokines, and other biologicals such as CTLA4-Ig. Steroids include, for example, prednisone and methylprednisone. NSAIDs include, for example, cyclo-oxygenase 1 (COX-1) inhibitors and cyclo-oxygenase 2 (COX-2) inhibitors. Immunosuppressive antibodies include both monoclonal and polyclonal antibody preparations, as well as engineered antibodies and antibody fragments, for example, OKT3, anti-CD25 (IL-2R alpha), and anti-IgE. Cyclosporines include cyclosporin A.

The term effective amount refers to the amount necessary or sufficient to realize a desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular active agent being administered, the size of the subject, or the severity of the disease or condition. One of ordinary skill in the art can empirically determine the effective amount of a particular active agent and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate system levels can be determined by, for example, measurement of the patient's peak or sustained plasma level of the drug. “Dose” and “dosage” are used interchangeably herein.

Generally, daily oral doses of active compounds will be from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

For any compound described herein the therapeutically effective amount can be initially determined from animal models. A therapeutically effective dose can also be determined from human data for active agents which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the active agent can be administered to a subject by any mode that delivers the active agent to the desired site. Administering the pharmaceutical composition of the present invention may be accomplished by any means known to the skilled artisan. Routes of administration include but are not limited to enteral, including but not limited to oral, and parenteral, including but not limited to intravenous, intramuscular, subcutaneous, intradermal, intranasal, sublingual, intratracheal, inhalation, ocular, vaginal, and rectal. Routes of administration also include direct injection, for example, intrasynovial and other intraarticular.

For oral administration, the compounds (i.e., active agents, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the active agent (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the active agent (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the active agent or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the active agents (or derivatives thereof). The active agent (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (α1-antitrypsin); Smith et al., 1989, J. Clin. Invest. 84:1145-1146 (α-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-γ and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of active agent (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified active agent may also be prepared in different formulations depending on the type of chemical modification or the type of device employed.

Formulations suitable for use with a nebulizer, either jet or ultrasonic, will typically comprise active agent (or derivative) dissolved in water at a concentration of about 0.1 to 25 mg of biologically active active agent per mL of solution. The formulation may also include a buffer and a simple sugar (e.g., for active agent stabilization and regulation of osmotic pressure). The nebulizer formulation may also contain a surfactant, to reduce or prevent surface induced aggregation of the active agent caused by atomization of the solution in forming the aerosol.

Formulations for use with a metered-dose inhaler device will generally comprise a finely divided powder containing the active agent (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing active agent (or derivative) and may also include a bulking agent, such as lactose, sorbitol, sucrose, or mannitol in amounts which facilitate dispersal of the powder from the device, e.g., 50 to 90% by weight of the formulation. The active agent (or derivative) should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The active agents and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of an active agent and optionally therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to the active agent, may be provided in particles. Particles as used herein means nano- or microparticles (or in some instances larger) which can consist in whole or in part of the active agent or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the active agent in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

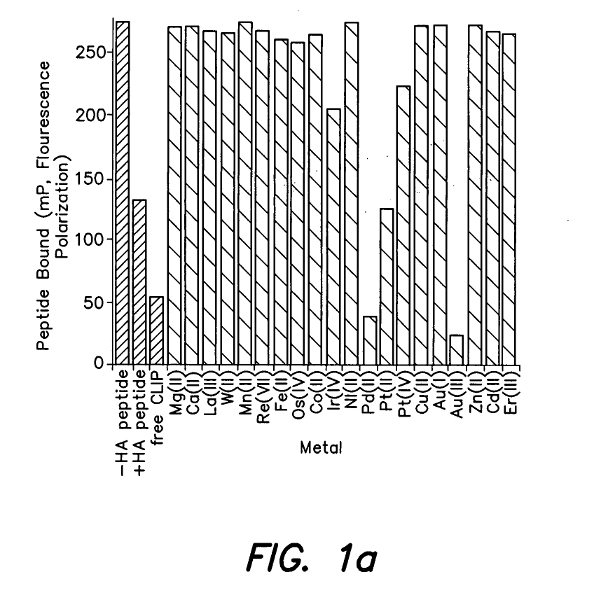

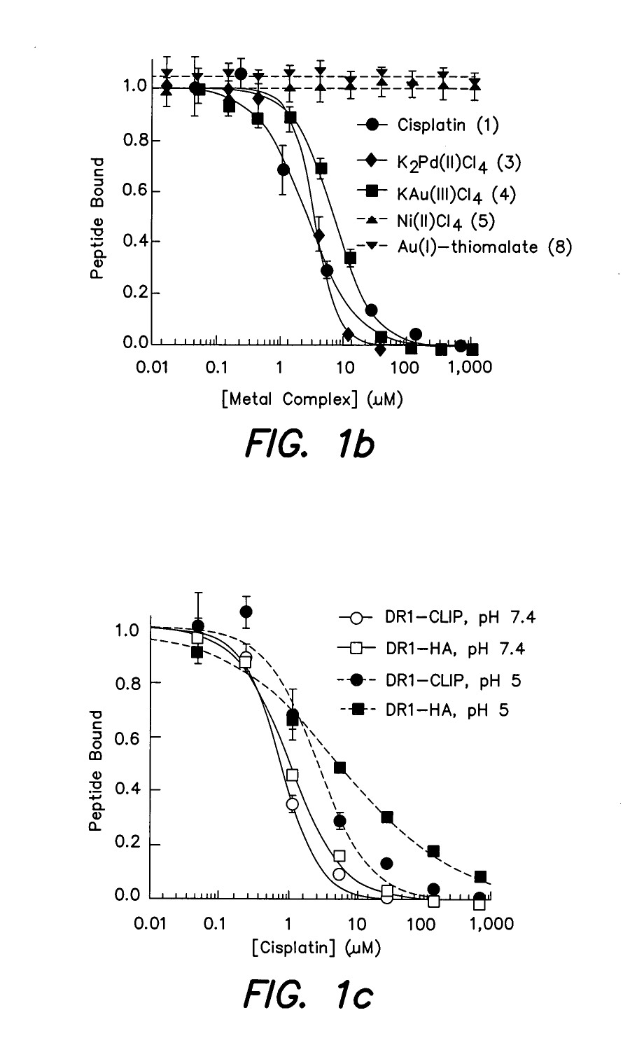

To find small-molecule modulators of class II MHC-peptide interactions, we used a high-throughput, fluorescence polarization assay to screen 28,000 compounds and natural extracts. Each well of a microplate contained a fluorescently labeled class II-associated invariant chain peptide (CLIP) bound to human leukocyte antigen (HLA)-DR1, a class II MHC protein. In addition, HLA-DM, a peptide exchange catalyst required in vivo for efficient loading, was present. The assay was performed at pH 5 to simulate endosomal pH (Morris P et al. (1994) Nature 368:551-4; Weber D A et al. (1996) Science 274:618-20). Compounds were added to the plates by pin transfer and monitored peptide release by fluorescence polarization. The screen uncovered only two active compounds, cisplatin (1) and carboplatin (2), both platinum(II) complexes. A focused screen of metal complexes for their ability to disrupt DR1-CLIP association was then carried out, results of which showed that complexes of two related noble metal ions, palladium(II) (K₂Pd^{II}Cl₄, 3) and gold(III) (KAu^{III}Cl₄, 4) were also active (FIG. 1a).

The IC₅₀ values for metal-induced peptide release were determined using a luminescence-based ELISA assay by titrating DR1-CLIP with the active metal complexes at pH 5. These values were similar for all active metals (IC₅₀, ˜1-10 μM; FIG. 1b). The active metal ions share several properties: they form square-planar, four-coordinate complexes and are isoelectronic (d⁸ electronic configuration). These metal ions are soft Lewis acids that favor coordination by soft Lewis bases (for example, the sulfur atoms found in cysteine (Kato M et al. (2005) J Chem Soc Dalton Trans. 6:1023-6), methionine (Isab A A et al. (1977) Biochim Biophys Acta 492:322-30) and cystine (Fakih S et al. (2003) Eur J Inorg Chem 6:1206-14) residues). In contrast, inactive Ni(II) (NiIICl₂; Kato M et al. (2005) J Chem Soc Dalton Trans. 6:1023-6), also a d⁸ metal, has a more intermediate Lewis acidity, and additional coordination geometries (for example, octahedral and tetrahedral) were observed (Richens D T (2005) Chem Rev 105:1961-2002). The ability of cisplatin to disrupt other peptide interactions was tested by measuring release of the hemagglutinin (HA) (306-318) peptide from DR1. The HA(306-318) peptide has a tenfold higher affinity for DR1 than CLIP4, yet it was released at similar concentrations (IC₅₀, ˜1 μM at pH 7.4 and ˜4 μM at pH 5.0; FIG. 1c). Similar results were obtained with KAu^{III}Cl₄ (discussed below). Therefore, the inhibitory activity is largely independent of peptide sequence and affinity as well as pH. These data demonstrate that square-planar metal complexes specifically and efficiently release peptide from DR1.

FIG. 1 shows that square-planar noble metal complexes release peptide from DR1. (a) A focused screen of metal complexes and salts showed that Pt(II), Pd(II) and Au(III) complexes were able to disrupt the MHC-peptide interaction. (b) Titration of DR1-CLIP complex by the active metal complexes, cisplatin (cis-Pt(NH₃)₂Cl₂), K₂PdCl₄ and KAuCl₄, and the inactive controls, NiCl₂ and sodium aurothiomalate (Au(I)-thiomalate). The calculated IC₅₀ values are 2.66±0.69 μM (cisplatin), 6.90±0.62 μM (KAuCl₄) and 3.48±0.37 μM (K₂PdCl₄). (c) Peptide displacement activity by cisplatin was essentially independent of peptide sequence or affinity as well as pH. At pH 5, the IC₅₀ values are 2.66±0.69 μM (CLIP) and 5.49±0.43 μM (HA). At pH 7.4, the IC₅₀ values are 0.77±0.03 μM (CLIP) and 1.06±0.14 μM (HA). Plots show mean ±s.d. of at least three replicates. c.p.s., counts per second.

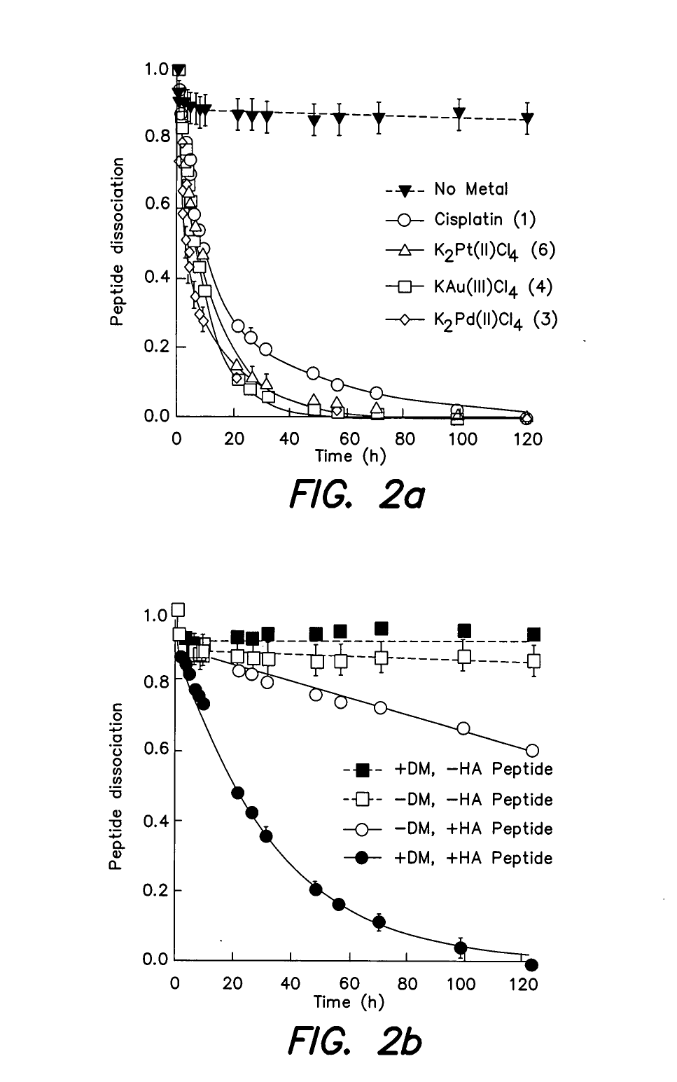

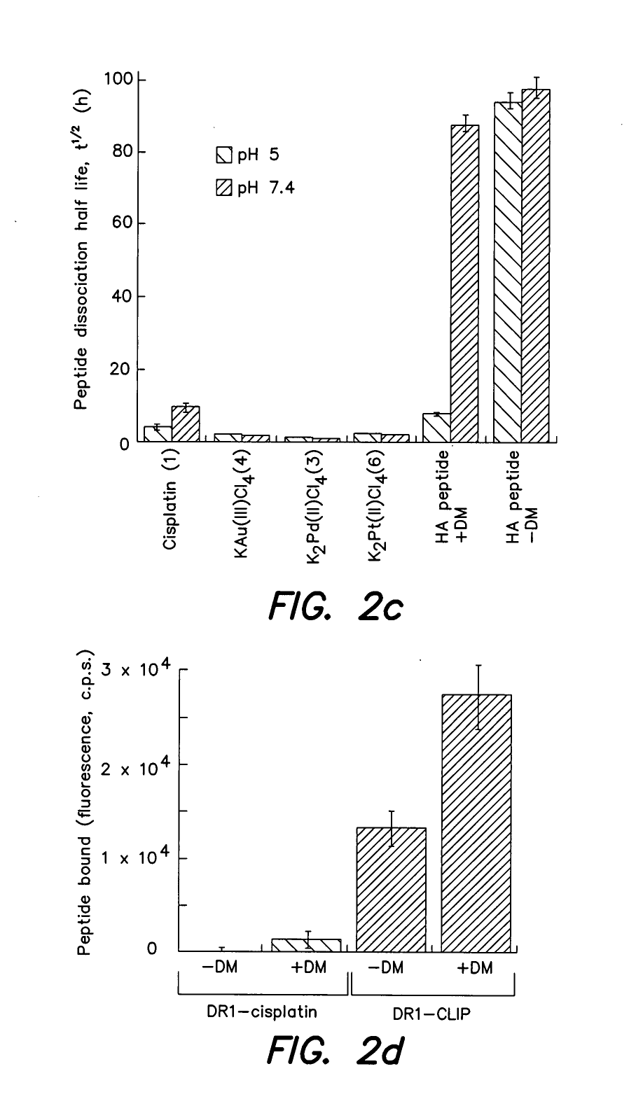

Peptide dissociation kinetics were measured in the presence of KAu^{III}Cl₄, K₂Pd^{II}Cl₄, K₂Pt^{II}Cl₄ (Isab A A et al. (1977) Biochim Biophys Acta 492:322-30) or cisplatin (FIG. 2a). The metal compounds triggered peptide dissociation in a DM-independent manner that was up to 60 times faster than the intrinsic dissociation rate (when excess unlabeled HA peptide was added) and five times faster than the rate measured when both unlabeled HA peptide and DM were present (FIG. 2a,b). A change in peptide dissociation rate of this magnitude (fivefold) could have significant biological effects, as a mere threefold increase in dissociation rate can render a peptide nonimmunogenic in mice (Lazarski C A et al. (2005) Immunity 23:29-40). The metal-mediated dissociation rate of CLIP is similar at both pH 5 and 7.4 (FIG. 2c); this is in contrast to DM-catalyzed peptide dissociation, which is fast at pH 5 and relatively slow at pH 7.4 (FIG. 2c) (Sherman M A et al. (1995) Immunity 3:197-205). Furthermore, unlike exchange catalyzed by DM, there seems to be no appreciable rebinding of peptide to DR1. As the metal-mediated peptide dissociation rate is faster than the intrinsic peptide dissociation rate, the metal complexes are not simple competitive inhibitors and must function by an allosteric mechanism. On the basis of these kinetic properties, the metal and peptide binding sites on DR1 must be distinct. This metal-binding site is a previously unknown allosteric site that, upon metal binding, induces a conformational change in DR1 that results in rapid peptide release.

To rule out the possibility that an interaction between the metal complex and peptide was responsible for peptide release, the ability of biotinylated HA peptide to rebind purified cisplatin-modified (and thus ‘peptide-empty’)DR1 (FIG. 2d) was tested. Pure complex was obtained by incubating DR1 with cisplatin until all peptide was released; then, the resulting DR1-cisplatin complex was dialyzed extensively and purified by gel filtration to remove excess cisplatin and unbound peptide. The relative kinetic stability of the DR1-cisplatin complex made purification possible. Pure Au(III) and Pd(II) complexes of DR1 were not obtained, as the protein precipitated from solution soon after removal of excess Au(III) or Pd(II). Metal complexes of Au(III) and Pd(II) have decreased stability relative to Pt(II) complexes (Richens D T (2005) Chem Rev 105:1961-2002), which, during the purification, could leave the protein in a destabilized, empty state that is highly prone to aggregation. Thus, the relative stability of the cisplatin complex allowed characterization of the metal-bound state of DR1. To determine the stoichiometry of this purified DR1-metal complex, the platinum concentration of the purified fraction was measured by inductively coupled plasma mass spectrometry. The DR1:Pt ratio in the purified complex was 1:1.2. Biotinylated HA peptide was then added to the stable DR1-cisplatin complex. Appreciable peptide binding was not observed, even in the presence of DM (FIG. 2d). These data demonstrate that the interaction between metal and DR1 is sufficient for the observed reduction of peptide affinity.

FIG. 2 illustrates that metal-mediated peptide dissociation kinetics and loss of peptide-binding activity by cisplatin-modified DR1 indicate that the metal complexes are noncompetitive inhibitors. (a) Metal-mediated peptide dissociation does not require DM or excess unlabeled HA peptide. The calculated half-lives (t_{1/2}) for CLIP dissociation were 4.34±0.75 h (cisplatin), 2.76±0.17 h (KAuCl₄), 1.59±0.24 h (K₂PdCl₄) and 3.05±0.15 h (K₂PtCl₄). There was essentially no peptide dissociation from DR1-CLIP alone (filled triangles). (b) The dissociation rate of CLIP was normally dependent on DM (unlabeled HA peptide was added to see complete dissociation). The t_{1/2} values with and without DM were 8.66±0.37 h and 94.6±1.97 h, respectively. When excess HA peptide was omitted, no dissociation was observed. (c) Peptide dissociation by the metal complexes was independent of pH, in contrast to the dissociation rate catalyzed by DM. (d) Purified DR1-cisplatin complex did not bind biotinylated HA peptide in the presence or absence of DM. For comparison, DR1-CLIP did exchange for HA peptide. Kinetic plots show mean ±s.d. of four replicates. The peptide-binding assay shows mean ±s.d. of eight replicates.

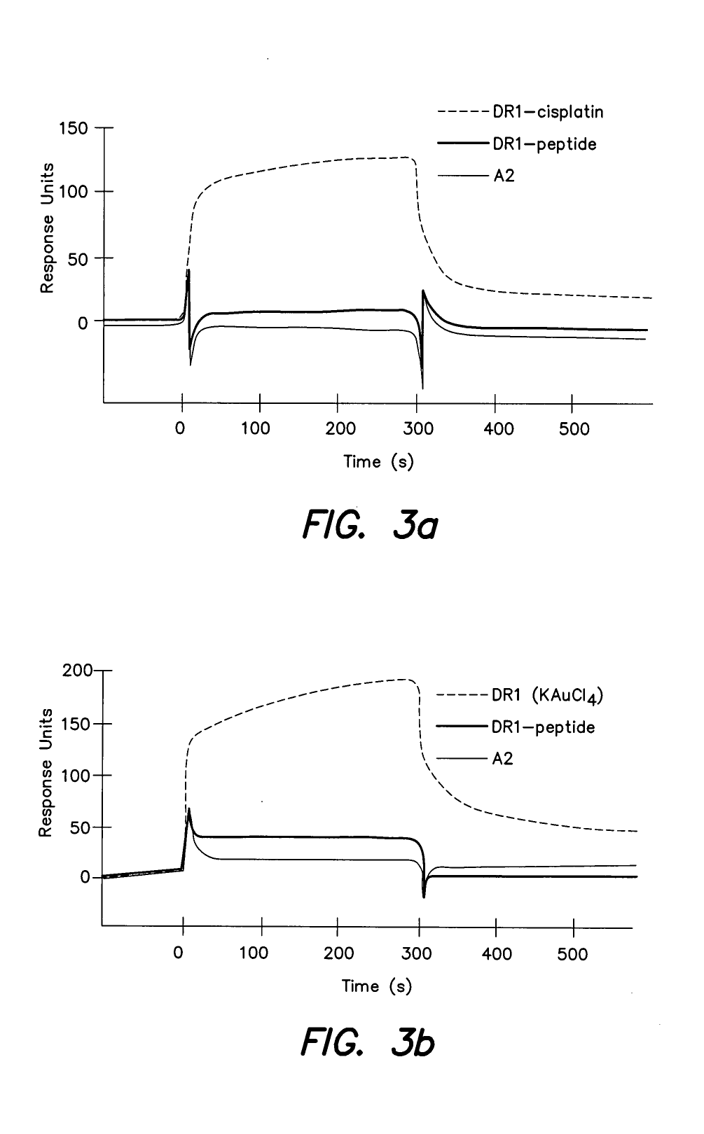

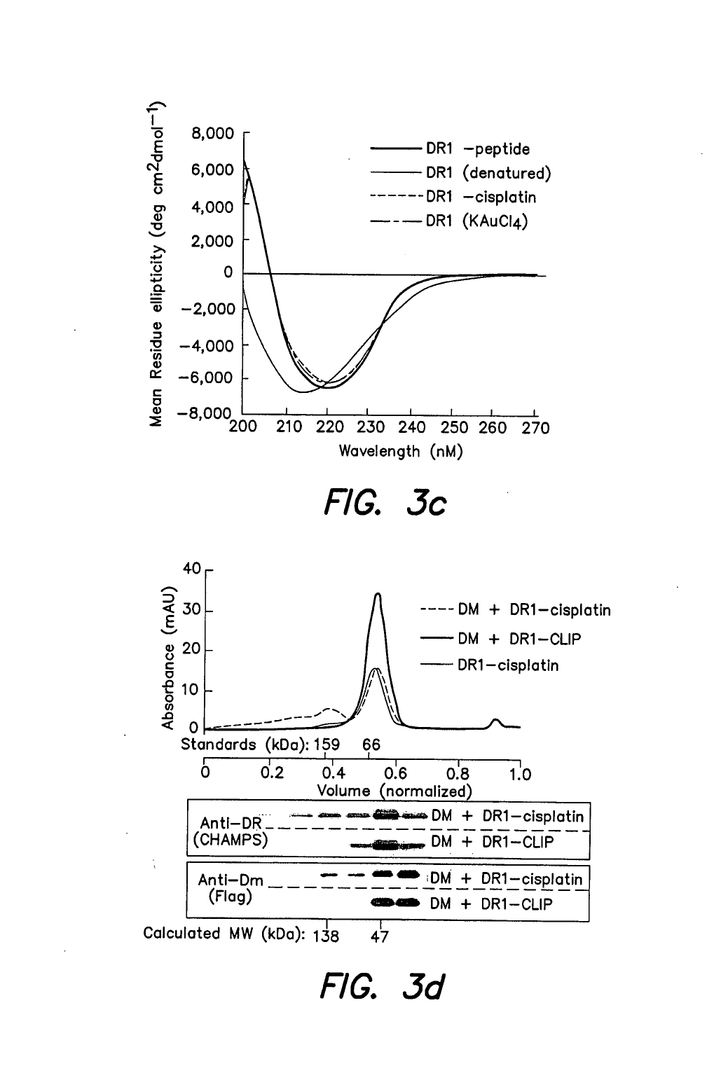

Further biochemical characterization of metal-modified DR1 demonstrated that the metal complexes induce a conformation of DR1 that is characteristic of a peptide-empty state. Using Biacore binding analysis, it was observed that the purified DR1-cisplatin complex bound to a monoclonal antibody (MEM-264) specific to a peptide-empty conformation of DR1 (FIG. 3a) (Carven G J et al. (2004) J Biol Chem 279:16561-70). Similarly, DR1-peptide that was prebound to the Biacore chip and then washed with KAuCl₄ (50 μM) also bound to MEM-264 (FIG. 3a). DR1-peptide complex and HLA-A2 (a class I MHC protein), both treated with KAuCl₄ (50 μM) and untreated, did not bind MEM-264. In addition, the circular dichroism spectrum of the purified DR1-cisplatin complex and DR1 incubated with KAuCl₄ (50 μM) were similar to native peptide-bound DR1 and differed substantially from denatured DR1 (FIG. 3b). These results show that metal binding to DR1 induces minimal secondary structural changes consistent with an empty native-like DR1.

Cisplatin-modified DR1 was tested to determine if it could bind to the peptide-exchange catalyst DM (FIG. 3c). Size exclusion chromatography of equimolar mixtures of DM and DR1-cisplatin (20 μM) showed a peak (138 kDa) that was not present when DM was mixed with DR1-CLIP (FIG. 3c). Western blot analysis of this peak confirmed that it contained both DM and modified DR1 (FIG. 3c, lower panel), and this peak ran at a position previously reported for a DR1-DM complex that was stabilized with a peptide covalently attached to DM12. Cisplatin-modified DR1 seems to stably associate with DM, but no such association occurs between DM and unmodified DR1, indicating that the DR1-cisplatin complex may resemble the transition state of the peptide-loading reaction.

FIG. 3 illustrates that metal-modified DR1 adopts a stable, peptide-empty conformational state that is recognized by DM. (a) Upper Panel: Purified DR1-cisplatin complex (biotin tagged) bound to a Biacore chip binds to the antibody MEM-264 that is specific for peptide-empty DR1 (top line). MEM-264 did not bind to peptide-loaded DR1 (middle line) or the class I MHC protein HLA-A2 (bottom line). Lower Panel: Biotin-tagged DR1-peptide complex bound to a Biacore chip and subsequently washed with KAuCl₄ also demonstrates MEM-264 binding activity (top line), whereas KAuCl₄-treated HLA-A2 does not (bottom line). (b) Circular dichroism spectra showed that the secondary structure of both purified DR1-cisplatin complex and DR1 incubated with KAuCl₄ was not denatured as compared with native DR1-peptide and denatured DR1. (c) Gel filtration chromatography indicated that DR1-cisplatin associates with DM (top curve). Gel filtration samples were also analyzed by immunoblotting with antibody to DR and DM (lower panels). The controls lack this higher-molecular weight peak, as shown by gel filtration, and lack DM and DR1 in the corresponding fractions, as shown by western blot (lower panel).

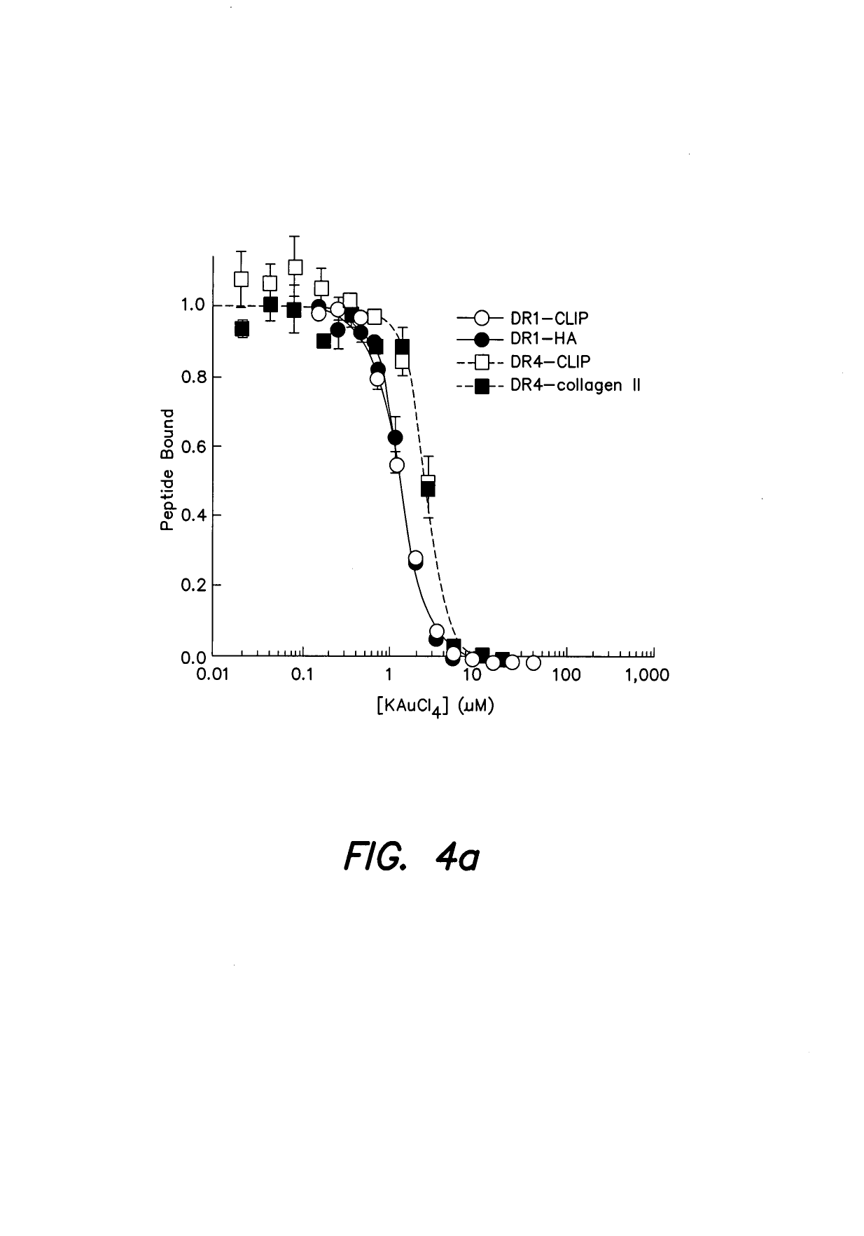

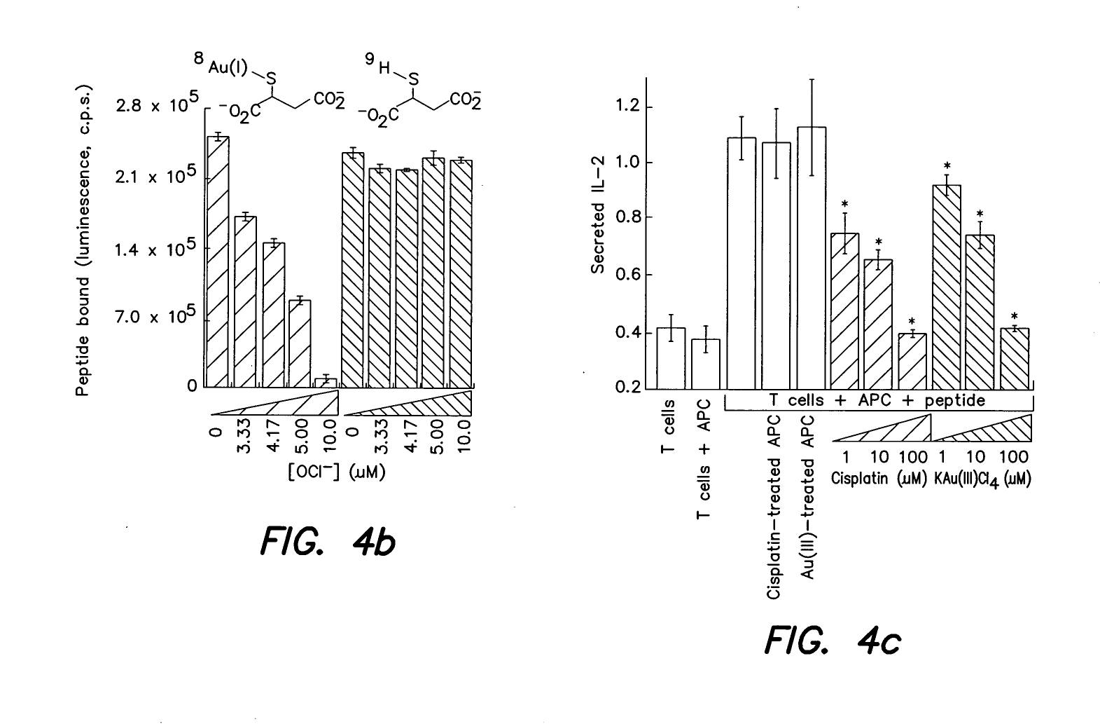

Complexes of gold(I) have been used for decades to treat rheumatoid arthritis (Kean W F et al. (1997) Br J Rheumatol 36:560-72), but gold(I) complexes (K[Au^{I}(CN)₂] (7) and sodium Au(I)-thiomalate (8)) did not release peptide from DR1 in vitro (FIG. 1a,b) (Griem P et al. (1995) J Immunol 155:1575-87; Takahashi K et al. (1998) Mol Immunol 35:1081-7). This result is consistent with previous studies that examined the effect of Au(I) compounds on antigen presentation (Griem P et al. (1995) J Immunol 155:1575-87; Takahashi K et al. (1998) Mol Immunol 35:1081-7). Yet it was found that Au(III) released collagen and CLIP peptides from HLA-DR4, a class II MHC allele frequently found in individuals with rheumatoid arthritis (FIG. 4a). The oxidation of Au(I) to Au(III) in vivo (Goebel C et al. (1995) Arch Toxicol 69:450-9) and in vitro (Shaw C F et al. (1994) Metal Based Drugs 1:351-62) has also been reported. Several research groups have demonstrated that the Au(III) metabolite formed in vivo was responsible for the severe allergic side effects that can occur from gold therapy (Goebel C et al. (1995) Arch Toxicol 69:450-9; Griem P et al. (1996) Eur J Immunol 26:279-87; Romagnoli P et al. (1992) J Clin Invest 89:254-8; Verwilghen J et al. (1992) Arthritis Rheum 35:1413-8). Based on these observations and the data presented herein, it is proposed that Au(I) complexes might be prodrugs, converted to the active Au(III) species at inflammatory sites by oxidative bursts of hypochlorite(OC1⁻) produced by phagocytes (Babior B M et al. (1973) J Clin Invest 52:741-4). Hypochlorite was tested to determine whether it could similarly activate the drug (sodium Au(I)-thiomalate) in the peptide release assay. Compound 8 and a control, thiomalate (9), were treated with increasing amounts of hypochlorite at pH 7.4 and added to DR1-HA (FIG. 4b). Mixtures of 8 and OC1⁻ caused peptide release, whereas mixtures of 9 and OC1⁻ did not (FIG. 4b). These data are consistent with a model in which Au(III) generated at inflammatory sites by the action of OC1⁻ could act to release peptide from MHC.

FIG. 4 illustrates that the in vitro inhibitory activity of these metal complexes correlates with their ability to block T cell activation. (a) KAu^{III}Cl₄ displaces peptides bound to DR1 and DR4, and the activity, as in the case of cisplatin (FIG. 1c), is independent of peptide sequence or affinity. The IC₅₀ values are 1.28±0.03 μM (DR1-CLIP), 1.13±0.14 μM (DR1-HA), 2.41±0.04 μM (DR4-CLIP) and 2.41±0.25 μM [DR4-human collagen II(261-273)]. (b) In vitro activation of sodium aurothiomalate (sodium Au(I)-thiomalate, 8) by hypochlorite (OC1⁻) solution causes peptide release from DR1. A control compound, thiomalate (9), does not have an effect when treated under identical conditions. (c) T-cell activation was blocked when antigen-presenting cells were pretreated with cisplatin or KAu^{III}Cl₄. As a control, activated B cells (untreated with metal complex) were mixed with metal-treated cells and presented to T cells. This mixture can activate T cells, ruling out any carry-through effect of the metal. Plots show mean ±s.d. of triplicates. *, P<0.05 (compared with untreated B cells).

A further set of experiments was performed to determine if the in vitro results would correlate with an in vivo T-cell activation assay. Previous work has indicated that fixed B cells pretreated with Au(III) are unable to activate T cells (Romagnoli P et al. (1992) J Clin Invest 89:254-8). The rapid rate of metal induced peptide release observed at both pH 5 and 7.4 suggested that these metal complexes may be able to disrupt peptide binding both in endosomes and at the cell surface (FIGS. 1c and 2c). Therefore, these metal complexes were tested to determine whether they were able to inhibit the activation of T cells using live B cells, LG-2, as antigen-presenting cells (APCs) (FIG. 4c). HA peptide-loaded APCs were incubated either in cisplatin or KAu^{III}Cl₄ solutions at concentrations similar to the IC₅₀ observed in vitro (1-100 μM) (FIG. 4c). Unbound metal was removed, APCs were presented to influenza-specific T cell hybridoma cells, and then cytokine release measured to test T-cell activation. Both metals showed significant inhibitory activity at concentrations that have been shown to be present in patients (Hashimoto K et al. (1992) J Clin Invest 89:1839-48). Cisplatin was more potent, possibly reflecting its slightly higher affinity for class II MHC proteins (FIG. 1b). In addition to a direct mechanism of peptide displacement, these compounds may also inhibit DM-mediated peptide loading by causing stable class II MHC-metal-DM complexes to form in the endosome (FIG. 3c), reducing the efficiency of antigenic peptide loading. Together, these data demonstrate that these noble metal complexes, cisplatin and KAu^{III}Cl₄, are able to disrupt peptide presentation by class II MHC both in vivo and in vitro.

Methods

All chemicals and solvents were purchased from commercial sources and used without further purification. Cisplatin and carboplatin were from Calbiochem (San Diego, Calif.). Sodium aurothiomalate, sodium hypochlorite, cisplatin, KAuCl₄, K₂PdCl₄, K₂PtCl₄, as well as all metal compounds used for the focused screen were purchased from Aldrich (St. Louis, Mo.) and thiomalate (D,L-mercaptosuccinic acid) from Acros (Morris Plains, N.J.). 100 mM Cisplatin and carboplatin stock solutions were made with dimethylsulfoxide (DMSO). All other metal stock solutions were prepared in deionized water.

Peptide synthesis, purification and labeling. All peptides were synthesized using solid phase chemistry. Purified Cys-containing peptides were labeled with either AlexaFluor 488 C5 maleimide (Molecular Probes) or EZ-Link PEO-maleimide activated biotin (Pierce). The peptides were purified by reverse phase HPLC on a preparative C18 column (Vydac). The mass and purity of each peptide was confirmed by LC/ESI-MS.

Peptide synthesis and purification. All peptides were synthesized on Wang resin using standard Fmoc chemistry. Cysteine residues were incorporated into the peptides in order to label them with maleimide activated probes (discussed below). The peptides were cleaved from the resin, deprotected, and purified by reverse-phase HPLC on a preparative C18 column (Vydac) using a gradient of 10-90% acetonitrile (with 0.1% TFA (v/v)). The mass and purity of each peptide was confirmed by LC/ESI-MS. The following peptides were used in these studies:

(SEQ ID NO:1)

CLIP(87-101), PVSKMRMATPLLMQA;

(SEQ ID NO:2)

CysCLIP(87-101), CPVSKMRMATPLLMQA;

(SEQ ID NO:3)

CLIP(87-101)-P5-Cys, PVSKMRMACPLLMQA;

(SEQ ID NO:4)

HA(306-318), PKYVKQNTLKLAT;

(SEQ ID NO:5)

CysHA(306-318), CPKYVKQNTLKLAT;

(SEQ ID NO:6)

Cys-human type II collagen(261-273) (CysCII),
CAGFKGEQGPKGEP.

All peptides were stored as solids at −20° C.

Protein expression, purification and peptide-loading. For binding and kinetic analysis, HLA-DR1, HLA-DR4 and HLA-DM were expressed in Drosophila melanogaster S2 cell lines and purified. For conformational analysis, proteins were expressed in Escherichia coli, refolded in presence or absence of peptide and purified. To prepare peptide complexes, purified DR1 or DR4 was incubated with a ˜20-fold molar excess of peptide for 4 d at 37° C. The peptide-loaded protein was subsequently purified by anion exchange chromatography followed by size exclusion chromatography.

Protein expression, purification, and peptide-loading. Soluble HLA-DR1, -DR4, and -DM were produced by Drosophila S2 cell lines and purified as described (Dessen A et al. (1997) Immunity 7:473-81; Sloan V S et al. (1995) Nature 375:802-6). HLA-DM was expressed with a FLAG epitope at the C terminus of the β chain. To prepare peptide complexes, purified DR1 or DR4 were incubated with a ˜20-fold molar excess of peptide (and 1 mM PMSF, 1 mM EDTA, and 0.02% sodium azide) for 4 days at 37° C. The solutions were filtered (Centrex MF-5, Schleicher & Schuell), diluted with 50 mM Tris-HCl (pH 8), purified by anion exchange chromatography (HiTrap Q HP, Amersham Biosciences). Class II MHC-peptide complexes were eluted from the column using a linear gradient from 0-40% 1 M NaCl (50 mM Tris-HCl, pH 8) followed by size exclusion chromatography (Superdex 200, Amersham Biosciences) in 20 mM HEPES, 100 mM NaCl (pH 7.4).

Preparation and purification of DR1-cisplatin complex. DR1-CLIP (0.1 μM) was diluted into pH 5.0 buffer (50 mM citrate, 150 mM NaCl) and incubated with cisplatin (0.5 to 1.0 mM) for 5 d at 37° C. After 5 d, the protein was dialyzed for 24 h at 4° C. against four exchanges of pH 5 buffer. Next, the protein was concentrated by centrifugal filtration, purified by size exclusion chromatography (20 mM HEPES, 100 mM NaCl, pH 7.4) and then concentrated (5-20 mM). The protein concentration was determined by measuring A₂₈₀ in 6 M guanidinium chloride (ε₂₈₀=61,275 M⁻¹cm⁻¹). Inductively coupled plasma mass spectrometry analysis of platinum content was performed by Robertson Microlit Laboratories.

Preparation and purification of DR1-cisplatin complex. DR1-CLIP complex was diluted to 0.1 μM in pH 5.0 buffer (50 mM citrate, 150 mM NaCl). A 33.3 mM (10 mg/ml) stock solution of cisplatin (Calbiochem) was prepared in DMSO and added to the DR1-CLIP solution to get a final cisplatin concentration of 1 to 0.5 mM. The solution was incubated for 5 days at 37° C. After 5 days, the protein was dialyzed (5 kDa MWCO dialysis tubing) against 2 l of pH 5.0 buffer (50 mM citrate, 150 mM NaCl) at 4° C. The buffer was exchanged 4 times over a period 24 hours. Next, the protein was concentrated by centrifugal filtration (10 kDa MWCO, Amicon Ultra-free, Millipore), and then purified by size exclusion chromatography (Superdex 200, Amersham Biosciences) (20 mM HEPES, 100 mM NaCl, pH 7.4), fractions were pooled, and concentrated. The protein concentration was determined by measuring A₂₈₀ in 6 M guanidinium chloride (ε₂₈₀=61,275 M⁻¹cm⁻¹) as described (Pace C N et al. (1995) Protein Sci 4:2411-23). The cisplatin-modified protein was stored at −80° C. Inductively coupled plasma mass spectrometry analysis of platinum content was performed by Robertson Microlit Laboratories, Inc., Madison, N.J.

General procedure for labeling Cys-containing peptides. Purified peptide (CysCLIP, CysHA, or CysCII) was dissolved in labeling buffer (20 mM HEPES, pH 7), or for less soluble peptides in a 50% DMF solution of labeling buffer (v/v). An equimolar amount of maleimide activated labeling reagent, either Alexa Fluor 488 C₅ maleimide (Molecular Probes) or EZ-Link PEO-maleimide activated biotin (Pierce), was dissolved in an equal volume of buffer and added to the peptide solution. The reaction flask was covered in aluminum foil and stirred at room temperature for 3 hours. Next, 2-mercaptoethanol was added (1 mol equiv.) to quench the unreacted maleimide labeling reagent and stirred for 30 minutes. The labeled peptide was purified by reverse-phase HPLC on a preparative C18 column (Vydac) using a 10-90% acetonitrile gradient. The mass and purity of each labeled peptide was confirmed by LC/ESI-MS.

Measurement of peptide release and dissociation kinetics. A chemiluminescent ELISA was used to measure the amount of bound biotin-labeled peptide that was released from DR in the presence of the metal complex. All metal complexes were dissolved in water, with the exception of cisplatin, which was dissolved in DMSO. The final DMSO concentration used in the binding and kinetic assays did not exceed 3.3% (v/v). Up to 3.3% DMSO had no effect on the activity of cisplatin or on the stability of class II MHC-peptide complexes. The effect of the metal complexes on the dissociation rate of AlexaFluor 488-labeled CLIP from DR1 was measured using the fluorescence polarization assay.

Fluorescence polarization (FP) based binding assay used for small molecule screening. A FP assay was developed in order to screen small-molecule libraries for chemicals that could modulate peptide binding by class II MHC proteins. The fluorescent DR1-CLIP-AlexaFluor488 (0.1 μM) was used for all FP studies. The concentration of DM used in the screening assay, and when included as a control, was 0.1 μM. Experiments were carried out in a final volume of 40 μl in black, 384-well assay plates (non-binding surface, Costar). The plate was sealed with an aluminum foil seal (Costar), placed in a sealed humidified container, and incubated at 37° C. The FP values for each experimental well were determined using a plate reader (Analyst, LJL Biosystems). A total of 27,937 compounds from several commercial sources were screened using this assay. This assay format was also used to screen several metal complexes and chloride salts as wells as for kinetic studies.

Focused screen of metal complexes using the FP assay. Based on the results of the primary small molecule screen, a focused screen consisting of several metal complexes and chloride salts was performed using the fluorescence polarization assay. The following metal salts and complexes were tested: CaCl₂, MgCl₂, CoCl₂, CdCl₂, MnCl₂, KAu(CN)₂, KAuCl₄, K₂PtCl₄, K₂PdCl₄, K₂PtCl₆, NiCl₂, FeCl₂, CuCl₂, ZnCl₂, K₂IrCl₆, Er(NO₃)₃, NH₄Re0₄, K₂OsCl₆, La(NO₃)₃, and K₄W(CN₆). These were tested in duplicate at a concentration of 250 μM. The results of this screen are shown in FIG. 1a. For the focused screen, DR1 (100 nM) bound to a Alexa-Fluor 488-labeled CLIP (af-CLIP) peptide was incubated with several metal complexes and salts (250 μM) for ˜20 h (37° C., pH 5.0), and then peptide release was measured by a fluorescence polarization assay. For comparison, the FP of labeled DR1-CLIP incubated with and without excess unlabeled HA peptide and the FP of af-CLIP peptide alone were also measured.

Measurement of peptide dissociation kinetics by metal complexes. The effect of the metal complexes on the dissociation of CLIP from DR1 was measured using the FP assay described herein. DR1-CLIP-Alexa Fluor488 complex (0.1 μM) was incubated at 37° C. in the presence of 100 μM HA peptide or 100 μM metal complex at pH 5 (50 mM citrate, 150 mM NaCl) and pH 7.4 (PBS). DR1-CLIP-af complex (100 nM) was incubated at 37° C. (buffer pH 5.0 or 7.4) in the presence active metal complex (100 μM) and the rate of peptide dissociation was measured by fluorescence polarization. For comparison, the control (DR1-CLIP) with no metal added was also tested to determine the background peptide release. Additionally, as controls the rate of CLIP dissociation in the with and without DM and excess HA peptide were also determined at pH 5 and 7.4. Each experimental condition was tested in quadruplicate in the presence or absence of DM (0.1 μM). The FP of the experimental wells was read at several time points over several days until the FP values reached baseline. The data were normalized and each point on the graph represents the mean of 4 independent experiments with the error bars representing the standard deviation.

Most reaction kinetic time courses were fitted by nonlinear regression to a single exponential process: Where nFP is normalized fluorescence polarization at time t; A is the amplitude of the phase and k is the rate constant.
nFP=A eˆ(−kt)
Extremely slow reactions without metal or peptide added required a fit to a double exponential process: Where nFP is normalized fluorescence polarization at time t; A1 and A2 are the amplitudes of both phases; k1 and k2 are the corresponding rate constants.
nFP=A1eˆ(−k1t)+A2eˆ(−k2t)
The regression analysis was performed by the commercial software package KaleidaGraph. Each data set was fit with a decay curve and the mean t_{1/2} and standard deviation was calculated from the 4 independent fits (FIG. 2).

Peptide binding by DR1-cisplatin complex. Purified DR1-CLIP or DR1-cisplatin complexes (0.1 μM of each) were incubated at 37° C. for 5 d with 1 μM HA peptide with and without DM (0.1 μM) in pH 5.0 buffer (50 mM citrate, 150 mM NaCl). After 5 d, the amount of peptide bound to DR1 was determined using a time-resolved fluorescence assay (DELFIA) following the manufacturer's guidelines (Perkin Elmer).

Peptide-binding by DR1-cisplatin complex. Purified DR1-CLIP or DR1-cisplatin complexes (100 nM) were incubated at 37° C., pH 5.0 for 5 days with biotin labeled HA peptide (1 μM) with and without DM (100 nM) present, and the amount of HA peptide bound to DR1 was determined using time-resolved fluorescence assay. The DELFIA format was used to measure the amount of biotin-HA that was bound to DR1. Briefly, after incubating for 5 days with biotin-HA peptide the DR1 was captured on a clear, 96-well plate (FluoroNunc, high-binding surface, NUNC) that was pre-coated with L243 (anti-DR antibody) and then blocked with 5% BSA (w/v) in PBST. Next, Europium-labeled streptavidin (Perkin Elmer) was added to detect bound biotin-HA. After washing with PBST, Enhancement Solution (Perkin Elmer) was added and the time-resolved fluorescence signal was measured using a microplate reader (Victor, Wallac).

Measurement of peptide release by metal complexes. Purified DR1 or DR4 (0.1 μM) loaded with a biotinylated peptide (biotin-CLIP, biotin-HA, or biotin-CII) were incubated overnight at 37° C. with increasing concentration of inhibitor or control compound in either pH 5.0 or pH 7.4 buffer without DM. The DR-biotin-peptide was captured on pre-blocked, streptavidin-coated 96-well plates (StreptaWell, Roche). Protein captured on the plate was detected by first adding anti-DR mAb L243 followed by an isotype matched IgG-POD antibody (Roche). After addition of a chemiluminescent substrate (ELISA POD substrate, Roche), the luminescence signal was read using a microplate reader (Analyst, LJL Biosystems).

The data were normalized to controls with and without labeled peptide. Each data point represents the mean of four independent experiments and the errors bars represent the standard deviation. IC₅₀ values were determined by nonlinear regression of peptide dissociation data by using the four-parameter logistic equation:
Response=Bottom+(Top−Bottom)/(1+(IC₅₀/[metal])ˆHillSlope)
The regression analysis was performed by the software package KaleidaGraph. The average IC₅₀s were calculated by independently fitting each set of titration curves with the dose-response curve and then calculating an average IC₅₀ and SD (FIG. 1).

Conformational analysis of DR1 complex. The conformation-specific monoclonal antibody MEM-264 was used to assess the conformation of metal-treated DR1. MEM-264 is specific for an epitope exposed on the empty but not the peptide-loaded form of DR1 (Carven G J et al. (2004) J Biol Chem 279:16561-70). The binding experiments were done on a BIAcore3000 using CM-5 carboxymethyldextran-coated gold chips coupled to modified avidin (Neutravidin). HLA-DR1 carrying a biotin tag on the C terminus of the b chain was either pretreated with cisplatin before attachment to the chip or attached first and then treated with a 5 min pulse of KAuCl₄ (50 μM) and washed for 5 min before antibody injection. For controls, the binding of MEM-264 to the DR1-peptide complex and biotin-tagged HLA-A2 (untreated and treated with 50 μM KAuCl₄) was also determined.

For circular dichroism analysis, proteins were concentrated to 15 μM and exchanged into PBS by spin ultrafiltration, and circular dichroism spectra (0.2 cm path length, Jasco spectropolarimeter) were obtained. KAuCl₄-treated (50 μM) DR1 was read with KAuCl₄ in solution, and denatured DR1 was produced by boiling the sample for 3 min before reading.

Gel filtration shift. Size exclusion chromatography was performed using the AKTA FPLC system (Amersham Biosciences) and a Superdex 200 column (Amersham Biosciences) at 4° C. The column was equilibrated with buffer (50 mM citrate, 150 mM NaCl, pH 5). Proteins were either mixed (20 μM of each) and injected immediately or injected separately (20 μM) in a final volume of 100 μl. Fractions (0.5 μl) were collected over the entire run and analyzed by western blot. Molecular weight standards were run to generate a standard curve.

Fractions from the gel filtration experiments were analyzed for the presence of DM and DR1 by western blot analysis. For detection of DM, we used anti-FLAG M2 (Sigma), and for DR1, we used polyclonal CHAMPS serum.

Conformational analysis of metal-modified DR1 by Biacore and CD. For comparison of the conformation of metal complexes of DR1 with empty and peptide-loaded DR1, proteins were prepared by in vitro folding of subunits expressed individually in E. coli as described (Frayser M et al. (1999) Protein Expr Purif 15:105-14). DR1 subunits were produced as inclusion bodies, purified under denaturing conditions by ion-exchange chromatography, and folded in the presence or absence of peptide as described (Frayser M et al. (1999) Protein Expr Purif 15:105-14). Refolded proteins were isolated by immunoaffinity or ion-exchange chromatography, for empty and peptide-loaded forms, respectively, followed by gel exclusion chromatography also as described (Frayser M et al. (1999) Protein Expr Purif 15:105-14). This protocol provides empty proteins free of potential contamination by adventitious peptides. Cisplatin complexes of the peptide-loaded form were prepared as described above for the Drosophila-expressed protein.

The empty state conformation of the metal treated protein was measured by binding to the antibody MEM-264 that is specific for an epitope exposed on the empty but not the peptide-loaded form of DR1 (Carven G J et al. (2004) J Biol Chem 279:1656-70). MEM-264 binding experiments were done on a BIAcore3000 using CM-5 carboxymethyldextran-coated gold chips. Modified avidin (Neutravidin) was coupled to the surface using standard NHS/EDC chemistry, yielding a binding signal of approximately 3000 RU. HLA-DR1 carrying a biotin tag on the C terminus of the β subunit (bioDR1-HA) (Cochran J R et al. (2000) Chem Biol 7:683-96) or HLA-A2 carrying a HIV-derived peptide and a biotin tag on the C terminus of the heavy chain and used as a control (bioA2) were immobilized to the neutravidin surface by 2-6 minute pulses of 50-100 μg/ml solutions in PBS using a flow rate of 10 μl/min; the exact conditions were chosen to yield 1000-2000 RU of bound MHC. bioDR1-cisplatin was prepared by incubation of bioDR1-HA with cisplatin as described above before immobilization. The bioDR1-Au(III) complex was prepared in situ first attaching bioDR1-peptide to the chip and then applying a 5 min pulse of KAuCl₄ (50 μM) followed by a 5 min wash immediately before antibody injection. As a control, the MHC class I protein bioA2 was given the identical KAuCl₄ treatment. The MEM-264 antibody was used as 0.5 mg/ml in PBS. All experiments were performed at 25° C. using a flow rate of 10 ul/min, and all traces shown were corrected for baseline changes by subtracting the response observed for the same solutions flowed in series over control flow cell containing only immobilized streptavidin.

For CD analysis, proteins were concentrated to 2 μM and exchanged into PBS by spin ultrafiltration, and CD spectra (Jasco spectropolarimeter) were obtained with a 0.2 cm path length. The CD spectrum of DR1 treated with KAuCl₄ (50 μM) was read with KAuCl₄ in the solution. For the denatured DR1 the DR1-HA complex was boiled 3 min prior to obtaining the CD spectrum.

Evidence that DR1-cisplatin forms a complex with DM.

Gel filtration experiment. The following standards were used: blue dextran (1×10³ kDa), ferritin (440 kDa), aldolase (158.8 kDa), bovine serum albumin (66 kDa), chicken egg albumin (45 kDa), carbonic anhydrase (29 kDa), α-lactalbumin (14.2 kDa), and vitamin B₁₂ (1.35 kDa). The retention volumes for each standard were normalized using the following equation:
K=(Vᵣ−V_{void})/(V_{void}−Vₜₒₜₐₗ);
where K is the normalized volume, Vᵣ is the retention volume of the sample, V_{void} is the void volume, and Vₜₒₜₐₗ is the total volume of the column. The void volume was 8.07 ml (the Vᵣ of blue dextran) and Vₜₒₜₐₗ was 20.66 mL (the Vᵣ of vitamin B₁₂). The normalized volume (K) for each standard was plotted versus the log(MW) and fit with a straight line to give the following equation:
K=2.15−0.3441 log(MW);
This equation was used to calculate the MW of the various protein species from their Vᵣ (see FIG. 3d).

Western Blot Analysis of Gel Filtration Fractions. Fractions from the gel filtration experiments were analyzed for the presence of DM and DR1. The fractions were boiled for 5 minutes in sample buffer (with 5% BME (v/v)), after first boiling the DR1-cisplatin samples were incubated at room temperature for 30 min and boiled a second time after adding an addition 5% BME, and the samples were resolved on a 4-20% SDS-PAGE gel (Criterion, Bio-Rad). Next, they were transferred overnight to a nitrocellulose membrane. After blocking the membrane with TBT (with 5% milk (w/v)) the primary antibodies were added. Monoclonal antibody anti-FLAG M2 (Sigma) was used for detection of DM. Polyclonal antibody CHAMPS was used for detection of DR1. After incubating with for 1 hour the membranes were washed and probed with IgG-POD (Roche).

Activation of sodium aurothiomalate (sodium Au(I)-thiomalate, 8) by sodium hypochlorite (NaOCl). Sodium aurothiomalate (8; 300 μM) and a control, thiomalate (D,L-mercaptosuccinic acid, 9), were dissolved in PBS and incubated for 1 h at 20° C. with increasing concentrations of NaOCl. After 1 h, 5 μl of each reaction stock solution was added to DR1-HA-biotin (0.1 μM) in PBS (pH 7.4). The final concentration of compounds 8 and 9 was 10 μM. After incubation of the protein with added compound overnight at 37° C., the amount of biotinylated HA peptide that bound to DR1 was determined by chemiluminescent ELISA.

Activation of sodium aurothiomalate (Au(I)-thiomalate, 8) by sodium hypochlorite (NaOCl). The concentration of sodium hypochlorite (NaOCl) solution was determined prior to each use by measuring the absorbance at 292 nm (ε₂₉₂=350 M⁻¹cm⁻¹). Sodium aurothiomalate (8), 300 μM, was incubated for one hour at 20° C. in the presence of increasing concentrations of NaOCl (0 μM, 100 μM, 125 μM, 150 μM, and 300 μM) in PBS (pH 7.4). As a control, thiomalate (D,L-mercaptosuccinic acid, 9), 300 μM, was also treated with the identical concentrations of NaOCl. After one hour, 5 μL of each reaction stock solution was added to 145 μL of DR1-HA-biotin complex in PBS (pH 7.4). The final protein concentration was 0.1 μM and the final concentration of compounds 8 and 9 was 10 μM. Final concentrations of oxidant were 0, 3.33, 4.17, 5 and 10 μM. Each concentration of oxidant was tested in quadruplicate. After incubating overnight at 37° C., the amount of biotinylated HA peptide that remained bound to DR1 was determined using the chemiluminescent ELISA described above.

T cell activation assay. HLA-DR1⁺Epstein-Barr Virus-transformed B lymphoblastoid LG-2 cells were incubated with 0.5 μM influenza HA peptide in serum-free AIM V medium (Invitrogen) for 2 h at 37° C. The peptide-containing medium was removed, and the antigen-presenting cells (APCs) were incubated with different concentrations of the metal compounds cisplatin or KAuCl₄ in PBS, pH 7.2 for 1.5 h at 37° C. The APCs were then washed and added at 50,000 cells per well with an influenza-specific T cell hybridoma, also at 50,000 cells per well, in a 96-well polystyrene plate. The assay was performed in S-MEM medium (Invitrogen) containing 10% FBS (HyClone). The cells were cultured for 16 h at 37° C., 5% CO₂. After incubation, the supernatants were analyzed for secreted interleukin-2 (IL-2) using an IL-2 ELISA (Peprotech).

T cell activation assay. HLA-DR1⁺Epstein-Barr Virus transformed B-lymphoblastoid LG-2 cells (Chicz R M et al. (1992) Nature 358:764-8) were incubated with 0.5 μM influenza peptide (Lamb J R et al. (1982) Nature 300:66-9) in serum-free AIM V medium (Invitrogen) for 2 hours at 37° C. The peptide-containing medium was removed, and the antigen-presenting cells (APCs) were incubated with different concentrations of the metal compounds cisplatin or potassium tetrachloroaurate (Au(III)) in PBS, pH 7.2 for 1.5 hours at 37° C. The APCs were then washed, and added at 50,000 cells per well with an influenza-specific T cell hybridoma (Boen E et al. (2000) J Immunol 165:2040-7), also at 50,000 cells per well, in a 96-well polystyrene plate. The assay was performed in S-MEM medium (Invitrogen) containing 10% FBS (HyClone). The cells were co-cultured for 16 hours at 37° C., 5% CO₂. After incubation, the supernatants were analyzed for secreted interleukin-2 (IL-2) using a mouse IL-2 ELISA (Peprotech).

Statistical analysis. All statistical analyses were performed using KaleidaGraph. For FIG. 4c, a two-tailed unpaired t-test was used; for cisplatin (1 μM), P=0.009355; for cisplatin (10 μM), P=0.001868; for cisplatin (100 μM), P=0.0001963; for KAu(III)Cl₄ (1 μM), P=0.04507; for KAu(III)Cl₄ (10 μM), P=0.0054; for KAu(III)Cl₄ (100 μM), P=0.0002382.

### REFERENCES

1. Watts, C. Capture and processing of exogenous antigens for presentation on MHC molecules. Annu. Rev. Immunol. 15, 821-850 (1997).
2. DeDecker, B. S. Allosteric drugs: thinking outside the active-site box. Chem. Biol. 7, R103-R107 (2000).
3. Morris, P. et al. An essential role for HLA-DM in antigen presentation by class II major histocompatibility molecules. Nature 368, 551-554 (1994).
4. Weber, D. A., Evavold, B. D. & Jensen, P. E. Enhanced dissociation of HLA-DR-bound peptides in the presence of HLA-DM. Science 274, 618-620 (1996).
5. Kato, M. et al. Inhibition of Thermus thermophilus HB8 thioredoxin activity by platinum(II). J. Chem. Soc., Dalton Trans. 6, 1023-1026 (2005).
6. Isab, A. A. & Sadler, P. J. Reactions of gold(III) ions with ribonuclease A and methionine derivatives in aqueous solution. Biochim. Biophys. Acta 492, 322-330 (1977).
7. Fakih, S. et al. Novel adducts of the anticancer drug oxaliplatin with glutathione and redox reactions with glutathione disulfide. Eur. J. Inorg. Chem., 1206-1214 (2003).
8. Richens, D. T. Ligand substitution reactions at inorganic centers. Chem. Rev. 105, 1961-2002 (2005).
9. Lazarski, C. A. et al. The kinetic stability of MHC class II:peptide complexes is a key parameter that dictates immunodominance. Immunity 23, 29-40 (2005).
10. Sherman, M. A., Weber, D. A. & Jensen, P. E. DM enhances peptide binding to class II MHC by release of invariant chain-derived peptide. Immunity 3, 197-205 (1995).
11. Carven, G. J. et al. Monoclonal antibodies specific for the empty conformation of HLADR1 reveal aspects of the conformational change associated with peptide binding. J. Biol. Chem. 279, 16561-16570 (2004).
12. Stratikos, E., Mosyak, L., Zaller, D. M. & Wiley, D. C. Identification of the lateral interaction surfaces of human histocompatibility leukocyte antigen (HLA)-DM with HLA-DR1 by formation of tethered complexes that present enhanced HLA-DM catalysis. J. Exp. Med. 196, 173-183 (2002).
13. Kean, W. F., Hart, L. & Buchanan, W. W. Auranofin. Br. J. Rheumatol. 36, 560-572 (1997).
14. Griem, P., Takahashi, K., Kalbacher, H. & Gleichmann, E. The antirheumatic drug disodium aurothiomalate inhibits CD4+ T cell recognition of peptides containing two or more cysteine residues. J. Immunol. 155, 1575-1587 (1995).
15. Takahashi, K., Kropshofer, H., Vogt, A. B., Gleichmann, E. & Griem, P. Drug-induced inhibition of insulin recognition by T-cells: the antirheumatic drug aurothiomalate inhibits MHC binding of insulin peptide. Mol. Immunol. 35, 1081-1087 (1998).
16. Goebel, C., Kubicka-Muranyi, M., Tonn, T., Gonzalez, J. & Gleichmann, E. Phagocytes render chemicals immunogenic: oxidation of gold(I) to the T cell-sensitizing gold(III) metabolite generated by mononuclear phagocytes. Arch. Toxicol. 69, 450-459 (1995).
17. Shaw, C. F. et al. Redox chemistry and [Au(CN)₂]— in the formation of gold metabolites. Metal Based Drugs 1, 351-362 (1994).
18. Griem, P., Panthel, K., Kalbacher, H. & Gleichmann, E. Alteration of a model antigen by Au(III) leads to T cell sensitization to cryptic peptides. Eur. J. Immunol. 26, 279-287(1996).
19. Romagnoli, P., Spinas, G. A. & Sinigaglia, F. Gold-specific T cells in rheumatoid arthritis patients treated with gold. J. Clin. Invest. 89, 254-258 (1992).
20. Verwilghen, J., Kingsley, G. H., Gambling, L. & Panayi, G. S. Activation of gold-reactive T lymphocytes in rheumatoid arthritis patients treated with gold. Arthritis Rheum. 35, 1413-1418 (1992).
21. Babior, B. M., Kipnes, R. S. & Curnutte, J. T. Biological defense mechanisms. The production by leukocytes of superoxide, a potential bactericidal agent. J. Clin. Invest. 52, 741-744 (1973).
22. Hashimoto, K., Whitehurst, C. E., Matsubara, T., Hirohata, K. & Lipsky, P. E. Immunomodulatory effects of therapeutic gold compounds. Gold sodium thiomalate inhibits the activity of T cell protein kinase C. J. Clin. Invest. 89, 1839-1848 (1992).
23. Koch, R. Uber Bacteriologische Forschung: Tenth International Medical Congress. Dtsch. Med. Wochenstr. 16, 756-757 (1890).

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A method for identifying a candidate immunosuppressive agent, comprising
contacting a peptide—class II MHC complex with a test agent;
measuring a test amount of peptide released from the contacted complex; and
identifying the test agent as a candidate immunosuppressive agent when the test amount exceeds a control amount of peptide released from the peptide—class II MHC complex in absence of the test agent.

2. A method for identifying a candidate immunosuppressive agent, comprising
contacting an uncharged class II MHC with a test agent;
contacting the uncharged class II MHC with a peptide;
measuring a test amount of uncharged class II MHC; and
identifying the test agent as a candidate immunosuppressive agent when the test amount exceeds a control amount of uncharged class II MHC in absence of the test agent.

3. The method of claim 2, wherein the contacting the uncharged class II MHC with the test agent precedes the contacting the uncharged class II MHC with the peptide.

4. The method of claim 2, wherein the contacting the uncharged class II MHC with the test agent is simultaneous with the contacting the uncharged class II MHC with the peptide.

5. The method of claim 2, wherein the measuring comprises measuring an amount of monoclonal antibody bound by the class II MHC, wherein the antibody is specific for a peptide-empty conformation of the class II MHC.

6. The method of claim 1 or claim 2, wherein the peptide is CLIP.

7. The method of claim 1 or claim 2, wherein the test agent is a composition comprising a d⁸ noble metal ion capable of forming a square-planar, four-coordinate complex.

8. The method of claim 1 or claim 2, wherein the test agent comprises Pd(II).

9. The method of claim 1 or claim 2, wherein the test agent comprises Pt(II) and excludes cisplatin.

10. The method of claim 1 or claim 2, wherein the test agent comprises Au(III).

11. A method for inhibiting an immune response, comprising
contacting APCs which express a class II MHC with a composition, other than cisplatin, comprising a d⁸ noble metal ion capable of forming a square-planar, four-coordinate complex, in an amount effective to inhibit peptide binding by the class II MHC.

12. The method of claim 11, wherein the d⁸ noble metal ion is Pd(II).

13. The method of claim 11, wherein the d⁸ noble metal ion is Pt(II).

14. The method of claim 11, wherein the d⁸ noble metal ion is Au(III).

15. A method of treating a subject having or at risk of developing an unwanted immune response, the method comprising
administering to the subject a composition, other than cisplatin, comprising a d⁸ noble metal ion capable of forming a square-planar, four-coordinate complex, in an amount effective to inhibit the unwanted immune response.

16. The method of claim 15, wherein the unwanted immune response is an autoimmune disease.

17. The method of claim 16, wherein the autoimmune disease is selected from the group consisting rheumatoid arthritis, systemic lupus erythematosus, insulin-dependent diabetes mellitus, multiple sclerosis, pemphigus vulgaris, gluten-sensitive enteropathy, and chronic active hepatitis.

18. The method of claim 15, wherein the unwanted immune response is allograft rejection.

19. The method of claim 15, wherein the unwanted immune response is an allergy.

20. The method of claim 15, wherein the unwanted immune response is allergic asthma.

21. The method of claim 15, wherein the unwanted immune response is inflammation.

22. The method of claim 15, wherein the administering is systemically administering.

23. The method of claim 15, wherein the administering is locally administering to a site involved in the unwanted immune response.

24. The method of claim 15, further comprising administering to the subject an immunosuppressive agent selected from the group consisting of steroids, non-steroidal anti-inflammatory drugs, immunosuppressive antibodies, cyclosporines, methotrexate, azathioprine, mycophenolate mofetil, tacrolimus, and sirolimus.

25. The method of claim 15, further comprising contacting the subject with an antigen.

26. The method of claim 25, wherein the contacting the subject with the antigen is administering the antigen to the subject.
